(19) United States
(12) Patent Application Publication
(10) Pub. No.: US 2012/0225093 A1
(43) Pub. Date: Sep. 6, 2012

(54) Abrogating HIV-1 Infection via Drug-Induced Reactivation of Apoptosis

(75) Inventors: Michael B. Mathews, Montclair, NJ (US); Hartmut M. Hanauske-Abel, Englewood Cliffs, NJ (US); Tsafi Pe'ery, Montclair, NJ (US); Mainul Hoque, North Arlington, NJ (US); Paul Palumbo, Lebanon, NH (US); Deepti Saxena, South Orange, NJ (US); Darlene D'Alliessi-Gandolfi, Harrison, NY (US); Myung-Hee Park, Potomac, MD (US)

(73) Assignee: UNIVERSITY OF MEDICINE AND DENTISTRY OF NEW JERSEY, Somerset, NJ (US)

(21) Appl. No.: 13/271,190

(22) Filed: Oct. 11, 2011

Related U.S. Application Data
(60) Provisional application No. 61/391,892, filed on Oct. 11, 2010.

(51) Int. Cl. A61K 31/4412; A61K 31/4418; A61P 37/04; C40B 30/04; A61P 31/18; C12Q 1/70; A61K 39/21

(52) U.S. Cl. 424/208.1; 435/5; 514/345; 514/348; 506/9

(57) ABSTRACT

The present invention relates to compositions and methods of treating, inhibiting, or controlling HIV infection.

### CROSS REFERENCE TO RELATED APPLICATION

This application claims priority of U.S. Provisional Application No. 61/391,892, filed on Oct. 10, 2010. The content of the application is incorporated herein by reference in its entirety.

### GOVERNMENT INTERESTS

The invention disclosed herein was made, at least in part, with Government support under Grant Nos HD-1457, AI034552 and AI060403 from the National Institutes of Health. Accordingly, the U.S. Government has certain rights in this invention.

### FIELD OF THE INVENTION

This invention relates to termination of HIV infection by, among others, medicinal apoptosis.

### BACKGROUND OF THE INVENTION

Human immunodeficiency virus (HIV) is a lentivirus that causes acquired immunodeficiency syndrome (AIDS), a condition in humans characterized by several clinical features including wasting syndromes, central nervous system degeneration and profound immunosuppression that results in life-threatening opportunistic infections and malignancies. Since its discovery in 1981, HIV type 1 (HIV-1) has led to the death of at least 25 million people worldwide. Great strides in behavioral prevention and medical treatment of HIV/AIDS notwithstanding, for the last several years the pandemic has claimed about 2.5 million lives annually (www.unaids.org) and remains unchecked. It is predicted that 20-60 million people will become infected over the next two decades even if there is a 2.5% annual decrease in HIV infections.

Studies of the HIV-1 life cycle led to the development of drugs targeting viral proteins important for viral infection, most notably reverse transcriptase and protease inhibitors. Despite the success of combinations of these drugs in highly active antiretroviral therapy (HAART), the emergence of drug-resistant HIV-1 strains, facilitated by the high mutation and recombination rates of the virus in conjunction with its prolific replication, poses a serious limitation to current treatments. Thus, there is a need for novel therapeutic agents and methods for treatment or inhibition of HIV infection.

### SUMMARY OF INVENTION

This invention relates to agents and methods for treating, inhibiting, or controlling HIV infection.

In one aspect, the invention features a method of identifying a compound for treating an infection with a virus. The method includes mixing a test compound with a first plurality of cells in a medium for a first period of time, the cells being infected with the virus; culturing the cells for a second period of time; and determining the activity level of the promoter of the virus in the cells. The activity level in the presence of the test compound, if lower than that in the absence of the test compound, indicates that the test compound is a candidate for treating the infection with the virus. In one embodiment, the culturing step includes (i) removing the test compound from the medium and (ii) maintaining the cells for the second period of time. The determining step can be conducted by determining the transcription initiation level. In one embodiment, the method further includes evaluating the apoptosis level of the cells; the level of apoptosis in the presence of the test compound, if higher than that in the absence of the test compound, indicates that the test compound is a candidate for treating the infection with the virus. The virus can be any virus of interest. Examples of the virus include retrovirus, such as an HIV-1 virus.

In one example, the first plurality of cells can be peripheral blood mononuclear cells (PBMCs). In that case, the above-described method further includes evaluating the level of IL-10 or IFN-γ in the medium or cells. The test compound is determined to be a candidate for treating the infection with the virus if the level of IL-10 or IFN-γ is decreases below or at a control level.

In another example, the above-described method further includes (a) contacting the first plurality of cells or the medium with a second plurality of cells, and (b) determining the activity level of the promoter of the virus in the second plurality cells in the same manner described above for determining whether the test compound is a candidate for treating the infection with the virus.

In the above-described method, the first period of time can be any duration between 2 hours and 1 month (e.g., between 12 and 132 hours, or between 24 and 48 hours). The second period of time can be any duration that is up to 3 months (e.g., between 12 hours and 3 months, or between 24 hours and 3 months).

In a second aspect, the invention features a method of reducing or eliminating HIV-1 rebound subsequent to treatment of an HIV-1 infected subject. The method includes administering an iron-chelating hydroxypyridinone (HOPO) compound to a subject infected with HIV-1 in an amount and for a time effective to reduce or eliminate the level of HIV-1 virions, followed by discontinuing administration of said iron-chelating hydroxypyridinone, whereby the level of HIV-1 virions remains reduced or eliminated for at least 4 weeks after discontinuation of administration. In one embodiment, the level of HIV-1 virions remains reduced or eliminated for at least 1, 2, 3, 4, 5, 10, 11, or 12 weeks after discontinuation of administration. The time effective to reduce or eliminate the level of HIV-1 virions can be 1-8 weeks, e.g., 4 weeks. In another embodiment, the method further includes administering to the subject an apoptosis inducer. The iron-chelating hydroxypyridinone can be one selected from the group consisting 6-cyclohexyl-1-hydroxy-4-methylpyrid-2(1H)-one (ciclopirox) and 3-hydroxy-1,2-dimethylpyridin-4(1H)-one (deferiprone).

In a third aspect, the invention features an immunogenic composition (e.g., a vaccine) containing (i) one or more cells that have been infected with a virus, e.g., HIV-1; (ii) an iron-chelating hydroxypyridinone compound; and (iii) a pharmaceutically acceptable carrier. Examples of the compound include ciclopirox (CPX) and deferiprone (DFE). The concentration of CPX can be between 1 and 100 μM (e.g., 3-70 μM, 10-50 μM, and 20-40 μM); the concentration of DFE can be between 1 and 100 μM (e.g., 10-500 μM, 50-400 μM, and 100-300 μM). In one embodiment, the cells are peripheral blood mononuclear cells (PBMCs). The immunogenic composition can further contain an adjuvant.

In fourth aspect, the invention features a method of eliciting an HIV-1-specific immune response in a subject. The method includes administering to a subject in need thereof (e.g., one that has been infected with HIV-1) the immunogenic composition mentioned above.

In fifth aspect, the invention features a method of increasing resistance to HIV-1 infection in a subject. The method includes (i) identifying a subject that has been, or is suspected of having been, or is expected to be, exposed to HIV-1, and (ii) administering to the subject an iron-chelating hydroxypyridinone in an amount and for a time effective to maintain or decrease the IL-10 or IFN-γ level in the subject. In one embodiment, the method further includes administering to the subject an apoptosis inducer. In another embodiment, the method further includes determining the IL-10 or IFN-γ level in the subject after the administering step.

The details of one or more embodiments of the invention are set forth in the description below. Other features, objects, and advantages of the invention will be apparent from the description and from the claims.

### BRIEF DESCRIPTION OF THE DRAWINGS

FIGS. 1A-D are a set of diagrams showing inhibition of HIV replication by drugs that block eIF5A modification. A. Hypusination of eIF5A (gray) occurs in two steps: the transfer, catalyzed by DHS, of an aminobutyl moiety (blue) from spermidine onto the side chain of eIF5A lysine-50, yielding deoxyhypusine (Dhp); and its subsequent hydroxylation, catalyzed by DOHH, yielding hypusine (Hpu). DHS is inhibited by GC7 and DOHH by CPX and DEF, as indicated. B. Structures of CPX, Agent P2, DEF and DFOX. C. CPX and DEF inhibit HIV replication in infected PBMCs. Infected PBMCs isolated from a single donor were co-cultured with uninfected PBMCs. CPX (30 μM), P2 (30 μM), or DEF (250 μM) were added 48 hr later. Amount of released p24 protein per million viable cells was determined every 24 hr. D. CPX and DEF inhibit gene expression from an HIV molecular clone in a dose dependant manner. The molecular clone pNL4-3-LucE- and pCMV-Ren were transfected into 293T cells and drugs were added to the concentrations shown. Dual luciferase assays were conducted at 12 hr post-transfection. Firefly (FF) luciferase expression was normalized to Renilla luciferase (Ren) from pCMV-Ren (mean of 2 experiments in duplicate, ±SD). Inset shows CPX and DEF effects on apoptosis and cell viability in untransfected 293T cultures as measured by staining with annexin V (AnnV) and 7-amino-actinomycin D (7AAD). Data are means of three time points (12, 18 and 24 hr) presented as percentages.

FIGS. 2A-E are a set of photographs showing ciclopirox and deferiprone prevent the maturation of eIF5A. A. Drug inhibition of eIF5A modification in 293T cells. Cells transfected with FLAG-tagged eIF5A were untreated or treated with increasing concentrations of CPX as indicated, or with agent P2. At 24 hr post-transfection, whole cell extract (WCE) was analyzed by immunoblotting with the NIH-353 anti-eIF5A antibody (upper panel) and anti-actin antibody (lower panel). B. Cells transfected with FLAG-tagged eIF5A were untreated or treated with increasing concentrations of DEF as indicated, or with DFOX. Cells were processed as in A. C. Cells transfected with FLAG-tagged eIF5A were treated with CPX (30 μM), P2 (30 μM), DEF (250 μM), DFOX (10 μM), or no drug (−). At 24 hr post-transfection, WCE was analyzed by immunoblotting with the NIH-353 anti-eIF5A antibody (upper panel) and anti-FLAG antibody (lower panel). The control culture was transfected with empty vector and no drug was added. D. Inhibition of enzyme-substrate binding. 293T cells transfected with FLAG-eIF5A were untreated (−) or treated with GC7 (10 μM) or CPX (30 μM), P2 (30 μM), DEF (250 μM), or DFOX (10 μM). WCE prepared at 24 hr post-transfection was immunoprecipitated with anti-FLAG antibody. Immunoprecipitates were immunoblotted with antibodies against DOHH (top panel) and FLAG (bottom panel). (*)-IgG light chain. E. 293T cells transfected with FLAG-DHS, FLAG-DOHH or empty vector (Control) were treated with GC7, CPX, or DEF, or no drug (−) at the same concentration as in panel D. Immunoprecipitates obtained with anti-FLAG antibody were immunoblotted and probed with anti-eIF5A antibody (BD). Input: WCE equivalent to 5% of the input was immunoblotted as a further control.

FIGS. 3A-D are a set of diagrams and photographs showing drug effects on luciferase expression from an HIV-1 molecular clone. A. Comparison of drug effects on luciferase expression from the pNL4-3-LucE- molecular clone in 293T cells. The molecular clone pNL4-3-LucE- and pCMV-Ren were transfected into 293T cells. Drugs were added where indicated at the following concentrations: P2 (30 μM), CPX (30 μM), DEF (250 μM), or DFOX (15 μM). Dual luciferase assays were conducted at 12 hr post-transfection. Firefly (FF) luciferase expression was normalized to Renilla luciferase (Ren) from pCMV-Ren (mean of 2 experiments in duplicate, ±SD). B. Expression in Jurkat cells was assayed essentially as in panel A. C. Firefly and Renilla luciferase RNA expression was analyzed in 293T cells treated as in panel A by RPA using 32P-[UTP] labeled antisense RNA probes corresponding to the C-termini of the FF and Ren luciferase mRNAs. D. Comparison of drug effects on p24 expression from the pNL4-3-LucE- molecular clone in 293T cells. Drugs were added where indicated to the same concentrations as in A. p24 levels were determined in cell extract at 12 hr post-transfection.

FIGS. 4A-C are a set of diagrams and photographs showing inhibition of HIV RNA expression from molecular clones. A. Schematic of HIV-1 provirus showing major transcripts, the position of the antisense probe, and fragments protected by RPA from spliced (S) and unspliced (U) transcripts. The positions of the Rev start codon mutation in pMRev(−) and the FF substitution in pNL4-3-LucE- are marked with one and two asterisks, respectively. B. Cytoplasmic and nuclear RNA isolated at 12 hr from 293T cells co-transfected with pNL4-3-LucE- and pCMV-Ren. Drugs were added where indicated at concentrations specified in FIG. 2D. RNA was isolated at 12 hr post-transfection. Autoradiograms display RPA fragments corresponding to HIV and Renilla RNAs (upper and middle panels, respectively). Renilla RNA was analyzed as in FIG. 3. The lower panel displays quantitation of protected spliced and unspliced RNA fragments relative to the Renilla RNA fragment (mean of 2 experiments in duplicate, ±SD). Probe: undigested probe in an amount equivalent to 5% of the input to the protection assays was run as a control. C. Effect of Rev. RNA from 293T cells transfected with the Rev-defective HIV molecular clone pMRev(−) together with (+) or without (−) Rev expression vector. RNA was isolated at 15 hr post-transfection. The lower panel displays quantitation of protected spliced and unspliced RNA fragments relative to the cytoplasmic unspliced control RNA (mean of 2 experiments in duplicate, ±SD).

FIGS. 5A-B are a set of diagrams showing sequence requirements for the drug sensitivity of the HIV molecular clone. A. Schematic of constructs expressing firefly luciferase from the CMV promoter (construct I, pCMV-FF) or the HIV promoter. Constructs III, IV and V were generated by deleting sequences from pNL4-3-LucE- (construct II). Construct VI was made by replacing the 3′LTR in construct V with the SV40 poly(A) sequence from pGL2TAR. Construct VII is a chimera of pGL2TAR and construct VI. B. CPX and DEF sensitivity of the constructs. Firefly luciferase expression from each construct was normalized to Renilla luciferase expression from pCMV-Ren as in FIG. 3, and presented as a percentage of the control ratio obtained in the absence of drugs.

FIGS. 6A-D are a set of diagrams and photographs showing inhibition of gene expression by CPX and DEF is promoter specific. A-C. Inhibition is independent of Tat. Total RNA was isolated 15 hr after transfection with pLTR-FF and pCMV-Ren in the absence or presence of Tat expression plasmid. Drugs were added as in FIG. 2D. RPA analysis was conducted by probing with antisense HIV-1 leader RNA probe complementary to LTR nt+83 to −117 (panel A). Protected fragments corresponding to promoter-proximal (Short) and promoter-distal (Long) transcripts were resolved (panel B) and quantified relative to Renilla RNA (panel C) analyzed as in FIG. 3. D. Stability of RNA transcribed from the HIV promoter in the presence of CPX. Actinomycin D (1 μg/ml) was added at 12 hr where indicated. RPA was carried out for FF mRNA as in FIG. 3. Upper panels: expression of FF RNA from the HIV promoter in control and CPX treated cells. Lower panel: FF mRNA decay rate in the presence or absence of CPX plotted relative to levels at 12 hr post-transfection (˜50% less in the presence of CPX).

FIGS. 7A-C are a set of diagrams and photographs showing depletion of eIF5A by siRNA inhibits gene expression from HIV-1 molecular clone. A. Depletion of eIF5A. 293T cells were transfected with 50 nM of eIF5A-1 siRNA (5A) or control siRNA (C, with no known complementary sequence in the human genome). Total RNA was isolated from transfected cells at the times indicated and analyzed by RPA using probes for eIF5A-1 or GAPDH mRNA (panel A). B. Effect of siRNA on HIV gene expression. siRNA-transfected cells were cotransfected with pNL4-3-LucE- and pCMV-Ren at 1, 3, 4, 5, 6 and 7 days after siRNA transfection and harvested 24 hr later for luciferase assays (top panel) as in FIG. 3. Relative FF/Ren luciferase expression at each time point is shown as a percentage inhibition of the control ratio (siC) obtained in the presence of si5A (triplicate measurements ±SD). Parallel cultures were analyzed for eIF5A and actin by immunoblotting (bottom panels). C. Lack of synergy between siRNA and drugs. siRNA transfected 293T cells were additionally transfected with pNL4-3-LucE- and pCMV-Ren 4 days later and simultaneously treated with CPX or DEF as indicated. Luciferase assays were analyzed as in FIG. 3. Immunoblots for eIF5A and actin are shown in the lower panels for days 3 and 4 after siRNA tranfection.

FIGS. 8A-F are a set of diagrams showing apoptotic activity of ciclopirox and deferiprone in uninfected and infected H9 cells. A-C. Apoptosis in H9-HIV cells treated with 30 μM CPX (circles) or 200 μM DEF (triangles) and in untreated controls (squares). The annexin V-positive and 7-amino-actinomycin D (7-AAD)-negative population was quantified by flow cytometry (A); cell diameter was quantified by image analysis (B); and live cells were quantified by computerized enumeration of trypan blue-stained samples (C). D. Mitochondrial membrane potential (ΔΨ collapse) and apoptotic proteolysis (89-kDa PARP accumulation) in H9-HIV cells (red) and uninfected H9 cells (blue). Assays were conducted by flow cytometry 24 hr after plating. Data (average±SEM) are calculated as percentage of cell population displaying ΔΨ collapse or 89-kDa PARP, and P values are indicated. E, F. Concentration-dependent degradation of mitochondrial membrane potential (ΔΨ collapse) in H9-HIV cells (red) and uninfected H9 cells (blue) treated for 24 hr with 30 μM CPX or 200 μM DEF. Results (average±SEM) were obtained by flow cytometry using 5,5′,6,6′-tetrachloro-1,1′,3,3′-tetraethylbenzimidazolcarbocyanine iodide (JC-1) and are expressed relative to untreated control cells. P values are indicated.

FIGS. 9A-C are a set of diagrams showing that ciclopirox increases apoptosis preferentially in HIV-infected H9 cells. A. Increased formation of the caspase-3-fragmented 89-kDa form of PARP in H9-HIV cells (red) and uninfected H9 cells (blue) after 24 hr of treatment with 30 μM CPX. Results (average±SEM) are presented as the fold-increase in PARP fragment-positive cells relative to untreated cells. B, C. Cell counts over the fluorescence intensity spectrum for 89-kDa PARP reactivity, quantified by flow cytometric single cell analysis after 24 hr (B) and 48 hr (C) of treatment with 30 μM CPX. Percentages of frag-PARP-positive H9-HIV (red) and uninfected H9 (blue) cells are calculated.

FIGS. 10A-C are a set of diagrams showing effects of ciclopirox on cellular and retroviral proteins in H9-HIV cells. A, B. Bcl-2 reactivity of HIV-infected (red) and uninfected (blue) H9 cells quantified by flow cytometry after 24 hr of treatment with CPX. A: Cell counts over the fluorescence intensity spectrum for Bcl-2 reactivity in cells treated with 30 μM CPX. B: CPX concentration dependence of Bcl-2 reactivity expressed as the geometric mean of fluorescence (average±SEM). C. Response of proteins in H9-HIV cells to 30 μM CPX after exposure for 24 hr (hatched bars) and 48 hr (filled bars). Retroviral and cellular proteins were labeled immunocytochemically and quantified in the same sample by flow cytometry. Data are presented as the geometric mean of fluorescence, normalized to time-identical infected untreated controls (100% values at 24/48 hr: p24, 36.2/38.2; trans-activator of transcription (Tat), 168.1/141.6; Rev, 7.8/6.4; viral protein R (Vpr), 1.7/1.7; activated caspase-3, 1.3/1.3). P values for deviation from respective controls are indicated: *=0.02; **≦0.004; ***≦0.0004.

FIGS. 11A-D are a set of diagrams showing drug-activated apoptosis and iron chelation. A. Covalent structures of the medicinal chelators DFOX and DEF, and of the antifungal agent CPX and its chelation homolog Agent P2. DFOX, CPX, and Agent P2 interact with iron via a hydroxamate moiety, similar to the chelating domain of DEF. Arrows indicate the uniform bidentate mode of metal binding. DFOX contains three of these moieties and is a hexadentate chelator. B. Effect of drugs and Agent P2 on the expression of iron-dependant (IRE; hatched bars) and retrovirally-encoded (HIV; filled bars) gene expression in transfected 293T cells. Results are expressed relative to untreated controls. C. Dose-dependent inhibition of deoxyhypusine hydroxylase activity in cells (HIV-1 infected H9 [H9-HIV]) by CPX (blue), but not by its chelation homolog Agent P2 (cyan). Triangles, peptide-bound hypusine; squares, peptide-bound deoxyhypusine. D. Induction of apoptosis by CPX and by DFOX. H9-HIV cells were treated for 24 hr and then assayed by flow cytometry using TUNEL. Results are expressed as percentage of cells that are TUNEL-positive (±SEM).

FIGS. 12A and B are a set of diagrams showing antiretroviral activity of ciclopirox in slow-onset infection of primary cells. Uninfected PBMCs from a single-donor were infected with isolate #990,135. Cultures were left untreated (open squares) or either CPX (red triangles) or Agent P2 (green circles) was added at 48 hr after plating/inoculation to 30 μM (small symbols) or 60 μM (large symbols). HIV-1 protein (p24; A) and copy number (HIV-1 RNA; B) were assayed at 24-hr intervals.

FIGS. 13A-E are a set of diagrams showing inhibitory action of ciclopirox in rapid-onset infection of primary cells. A-C. Blockade of acute HIV-1 infection and activation of HIV-enhanced apoptosis. Uninfected PBMCs from a single-donor were cultured without infection (open symbols) or were infected with 58,500 copies/ml of HIV-1 isolate #990,010 (filled symbols). After 12 hr, CPX was added to 30 μM (open squares) or cultures were left untreated (triangles). HIV-1 p24 (A) and RNA (B) were assayed at intervals and apoptotic cells were enumerated by TUNEL (C). Active retroviral gene expression occurs in Phase I, preceding suppression of apoptosis in Phase II (green line segments). In CPX-treated infected cultures, retroviral gene expression is inhibited in Phase I and apoptosis is activated in Phase II (red line segments). D, E. Response of innate cytokines. Cells were treated as above, except that CPX addition was coincident with infection. IFN-γ (D) and IL-10 (E) were analyzed during Phase I in the same samples by flow cytometric bead assay. Values are the mean of two independent experiments (initial levels in pg per 10⁶ vital cells for HIV-exposed CPX-treated/HIV-exposed untreated/uninfected CPX-treated cells: IFN-γ, 657/209/634; IL-10, 34/13/40).

FIG. 14 is a diagram showing long-term suppression of HIV-1 infection in PBMC cultures by ciclopirox. Multiple-donor PBMC cultures were infected with isolate #990,010 and replenished with fresh cells and medium as indicated by arrowheads; on each occasion, half of the culture was replaced. After a one-week period (1) to establish infection ex vivo, the culture was treated with 30 μM CPX for one month (2), then the drug was withdrawn (asterisk) and the culture was assayed for HIV-1 protein and viral copy number over a subsequent three-month period (3) to monitor for re-emerging productive infection (Phase III). p24 assays: open circle, HIV-exposed untreated cultures; closed circles, HIV-exposed cultures, treated with CPX. HIV-1 RNA assays: open squares, HIV-exposed untreated cultures; closed triangles, HIV-exposed cultures during CPX treatment; open triangles, HIV-exposed cultures after withdrawal of CPX. Arrows a and b denote the detection limits of the p24 and HIV-1 RNA assays, respectively. Due to the continuous replenishment with freshly isolated uninfected PBMCs, the viability of cultured cells was consistently above 90% as assessed by computerized vital dye exclusion.

FIG. 15 is a diagram showing integrity of human uterine epithelial cultures treated with deferiprone. Time course of epithelial barrier function, measured as transepithelial resistance (±SEM) of confluent ECC-1 cells. Black circles, control; green triangles, 20 μM DFOX; cyan circles, 200 μM DEF. P values for DEF-treated vs. control cultures are shown.

FIG. 16 is a diagram showing treatment of mouse vaginal mucosa with the gynecological preparation of ciclopirox. A, B: histology of vaginal mucosa of medroxyprogesterone-synchronized mice, untreated (A) or intravaginally treated (B) for four consecutive days with the antifungal gynecological formulation of CPX (1% Batrafen Vaginalcrème™). A1 and B1, stained with hematoxylin-eosin; A2 and B2, stained with anti-active caspase-3. Due to the progestin synchronization of all animals, the vaginal mucosa of untreated (A) and treated (B) animals displays a luminal surface of living cuboidal mucinous cells, overlying uncornified strata of living squamous epithelial cells. C, D: tissue reactivity to anti-active caspase-3 for two organs known to contain cells undergoing apoptosis, human neonatal thymus (C) and mouse ovary (D1-D3). Active caspase-3 locates to the nuclei of cortical lymphocytes and folliculogenic cells, respectively, consistent with its established nuclear occurrence, and generates a characteristic, punctate staining pattern. Batrafen-treated vaginal mucosa does not display this apoptotic pattern (B2), showing instead the faint cytoplasmic reactivity of untreated controls (A2). The images of B2, evidencing absence of apoptotic cells after vaginal Batrafen exposure, and of D1-D3, evidencing presence of physiologically apoptotic cells in the ovary, were taken from the same longitudinal cut that sections an animal's entire reproductive tract.

FIG. 17 is a diagram showing a model for the antiretroviral mechanism of ciclopirox and deferiprone via “therapeutic reclamation of apoptotic proficiency” (TRAP). The model is based on cellular proapoptotic activation (1) and viral antiapoptotic deactivation (2), the latter conceived as a composite of increased viral proapototic and decreased viral antiapoptotic factors. Uninfected cells may suffer a decrease in their apoptotic threshold due to (1), but in the absence of (2), they largely escape catastrophic completion of intrinsic pathway activation. Yellow boxes, cellular events; green boxes, viral events

### DETAILED DESCRIPTION OF THE INVENTION

This invention is based, at least in part, on unexpected discoveries that iron-chelating hydroxypyridinone (HOPO) compounds (such as ciclopirox (CPX) and deferiprone (DEF)) effectively blocked retroviral gene expression and acted by therapeutic reclamation of apoptotic proficiency in HIV-1 infected cells.

It was known in the art that HIV-1 evades the innate and adaptive responses of the immune system, and exploits both to its advantage. In susceptible cells, HIV-1 establishes infection that resists clearance by all currently known therapies. A major feature of this resistance is interference with programmed cell death (apoptosis), a primal protection of cells against viral invasion and persistence.

After HIV-1 entry, apoptosis remains functional for a brief period. Yet, marked resistance to proapoptotic stimuli occurs in HIV-infected cell lines and cultured primary cells, but not their uninfected counterparts, mediated by retroviral proteins and miRNAs. In brain and blood, infected monomyelocytic cells are protected against apoptosis. Their stable antiapoptotic gene expression secures viability as mobile infective units and long-term reservoirs. Only 0.1% of productively infected cells in lymph nodes become apoptotic. Furthermore, HIV-1 re-programs susceptible cells to kill uninfected “bystanders,” resulting in extensive apoptosis of HIV-specific cytotoxic lymphocytes. T cell depletion, due to virally promoted apoptotic death of uninfected and infected cells, eventually causes immune deficiency.

The prominent role of apoptosis in HIV/AIDS was recognized early (Gougeon et al. (1991) C R Acad Sci III 312: 529-537; Groux et al. (1991) C R Acad Sci III 312: 599-606; and Montagnier L (2009) Angew Chem Int Ed Engl 48: 5815-5826), suggesting that inhibitors of apoptosis could be combined with antiretrovirals to preserve immune system function by promoting the survival of ‘bystander’ cells (13. Finkel et al. (1995) Nat Med 1: 129-134; Selliah et al. (2001) Cell Death Differ 8: 127-136; and Soldani et al. (2002) Apoptosis 7: 321-328). Surprisingly, the studies reported herein support an alternative approach, namely the use of activators of apoptosis for the ablation of pathogenic HIV-infected cells that destroy the immune system.

As disclosed herein, assays were to examine the activity of two hydroxypyridinone compounds and it was shown that they inhibited HIV-1 gene expression in cellular models and HIV-1 replication in infected PBMCs cultured ex vivo. Ciclopirox (CPX; 6-cyclohexyl-1-hydroxy-4-methylpyridin-2[1H]-one: e.g., Batrafen™, Dafnegin™) is a well-tolerated topical fungicide used in gynecological and dermatological preparations, and deferiprone (DEF; 3-hydroxy-1,2-dimethylpyridin-4(1H)-one: e.g., Ferriprox™) is a systemically active medicinal chelator administered orally mostly to thalassemic patients. Chemically, both drugs are iron-chelating hydroxypyridinones (HOPOs), classified among the 1,2- and 3,4-HOPOs, respectively (Scott et al. (2009) Chem Rev 109: 4885-4910). Biochemically, both drugs inhibit protein hydroxylation by the 2-oxoacid utilizing class of non-heme iron dioxygenases (Clement et al. (2002) Int J Cancer 100: 491-498; Hanauske-Abel et al. (2003) Curr Med Chem 10: 1005-1019; and McCaffrey et al. (1995) J Clin Invest 95: 446-455) at clinically relevant concentrations.

In addition, the drugs are potent inhibitors of the hydroxylation of eukaryotic translation initiation factor 5A (eIF5A), a cellular protein involved in both apoptosis and HIV-1 replication. DEF and CPX inhibit HIV-1 gene expression at the level of transcript initiation (Hogue et al. (2009) Retrovirology 6: 90), potentially disrupting viral control over cellular apoptosis as well as the expression of HIV-1 genes essential for acute infection. Consistent with this, DEF was shown to trigger apoptosis in a latently HIV-infected cell line after mitogen stimulation, but not in its uninfected parent, although the underlying mechanism was not established. As disclosed herein, assays were carried out to address the generality of this action, define its mechanism, assess its ability to clear HIV-1 infection, and examine the tolerance of epithelial cells to the drugs.

As disclosed herein, both CPX and DEF overcome retrovirally-induced resistance to apoptosis and activate apoptosis selectively in a chronically HIV-infected CD4+ T cell line and in infected PBMCs. The apoptotic mechanism is triggered through the intrinsic mitochondrial pathway. Prior to apoptosis, the drugs suppress acute infection of PBMCs exposed to patient-isolated HIV-1. Notably, self-sustaining HIV-1 infection of long-term PBMC cultures is effectively cleared by the drugs and productive infection does not return after drug removal—i.e., there is no rebound. Also, it was found that the drugs, which are prescribed safely for indications unrelated to HIV-AIDS, caused no deleterious effects in sensitive human tissue culture and mouse in vivo models of epithelial cell integrity, even at very high concentrations. The results disclosed herein indicate that the underlying mechanism for these effects is inhibition of protein hydroxylation and that this hydroxylation is a novel target to achieve antiretroviral effects. The results further suggest that CPX and DEF, as well as other hydroxypyridinone compounds, can be used in treating HIV infection.

### Drug Screening

The invention provides a method for identifying a compound that inhibits viral transcriptional gene expression, viral interfering RNAs, viral proteins, or productive infection of a virus (e.g., HIV). The compound thus-identified can be used to treat an infection with the virus.

Candidate compounds to be screened (e.g., proteins, peptides, peptidomimetics, peptoids, antibodies, small molecules, or other drugs) can be obtained using any of the numerous approaches in combinatorial library methods known in the art. Such libraries include: peptide libraries, peptoid libraries (libraries of molecules having the functionalities of peptides, but with a novel, non-peptide backbone that is resistant to enzymatic degradation); spatially addressable parallel solid phase or solution phase libraries; synthetic libraries obtained by deconvolution or affinity chromatography selection; and the “one-bead one-compound” libraries. See, e.g., Zuckermann et al. 1994, J. Med. Chem. 37:2678-2685; and Lam, 1997, Anticancer Drug Des. 12:145. Examples of methods for the synthesis of molecular libraries can be found in, e.g., DeWitt et al., 1993, PNAS USA 90:6909; Erb et al., 1994, PNAS USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994 J. Med. Chem. 37:1233. Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (U.S. Pat. No. 5,223,409), spores (U.S. Pat. No. 5,223,409), plasmids (Cull et al., 1992, PNAS USA 89:1865-1869), or phages (Scott and Smith 1990, Science 249:386-390; Devlin, 1990, Science 249:404-406; Cwirla et al., 1990, PNAS USA 87:6378-6382; Felici 1991, J. Mol. Biol. 222:301-310; and U.S. Pat. No. 5,223,409).

To identify an inhibitor mentioned above, one can contact a candidate compound with a system containing cells that have been infected with a virus or recombinant cells that contain a reporter gene under the control of the promoter of the virus. The system can be an in vitro cell line model or an in vivo animal model. The cells can naturally express the viral gene, or can be modified to express a recombinant nucleic acid. The recombinant nucleic acid can contain a nucleic acid coding a reporter polypeptide to a heterologous promoter. One then measures the expression level of the reporter polypeptide or viral protein. The expression level can be determined at either the mRNA level or at the protein level.

Methods of measuring mRNA levels in a cell, a tissue sample, or a body fluid are well known in the art. To measure mRNA levels, cells can be lysed and the levels of mRNA in the lysates or in RNA purified or semi-purified from the lysates can be determined by, e.g., hybridization assays (using detectably labeled gene-specific DNA or RNA probes) and quantitative or semi-quantitative RT-PCR (using appropriate gene-specific primers). Alternatively, quantitative or semi-quantitative in situ hybridization assays can be carried out using tissue sections or unlysed cell suspensions, and detectably (e.g., fluorescent or enzyme) labeled DNA or RNA probes. Additional mRNA-quantifying methods include RNA protection assay (RPA) and SAGE. Methods of measuring protein levels in a cell or a tissue sample are also known in the art.

To determine the ability of a candidate compound to inhibit the viral transcriptional gene expression or others mentioned above, one can compare the level obtained in the manner described above with a control level or activity obtained in the absence of the candidate compound. If the level is lower than the control, the compound is identified as being effective for treating the disorders mentioned above. One can further verify the efficacy of a compound thus-identified using the in vitro cell culture model or an in vivo animal model as disclosed in the example below.

The invention also provides a method for identifying a compound that increases apoptosis level in the above-mentioned cell system. The compound thus-identified can also be used to treat an infection with the virus.

The level of apoptosis in the cells brought into contact with the test compound can be evaluated by many methods known in the art and those disclosed herein. For example, agarose gel electrophoresis for detecting a fragment cleaved in DNA nucleosome units as a “DNA ladder,” pulse field electrophoresis for detecting apoptosis that produces 50 to 300 kbp high molecular weight DNA fragments, the in situ end labeling method (TUNEL method) for detecting a DNA cleavage end is to detect apoptosis in tissue, and a method comprising staining cells with a fluorescent dye, and thereafter performing the detection of cell size change and cells with decreased DNA contents or detection of live or dead cells, and the like, by flow cytometry, and the like can be used. See e.g., US Application Nos. 20110124523, 20100297145, and 20100215638.

### Treatment

The invention provides a composition that contains a suitable carrier and one or more of the active agents described above, e.g., CPX and DEF. The composition can be a pharmaceutical composition that contains a pharmaceutically acceptable carrier. The term “pharmaceutical composition” refers to the combination of an active agent with a carrier, inert or active, making the composition especially suitable for diagnostic or therapeutic use in vivo or ex vivo. The term “pharmaceutically acceptable carrier” refers to any of the standard pharmaceutical carriers, such as a phosphate buffered saline solution, water, emulsions, and various types of wetting agents. The compositions also can include stabilizers and preservatives. A pharmaceutically acceptable carrier, after administered to or upon a subject, does not cause undesirable physiological effects. The carrier in the pharmaceutical composition must be “acceptable” also in the sense that it is compatible with the active ingredient and, preferably, capable of stabilizing it. One or more solubilizing agents can be utilized as pharmaceutical carriers for delivery of an active agent. Examples of other carriers include colloidal silicon oxide, magnesium stearate, cellulose, and sodium lauryl sulfate.

Pharmaceutically effective compositions of this invention may be administered to humans and other animals by a variety of methods that may include continuous or intermittent administration. Examples of methods of administration may include, but are not limited to, oral, rectal, parenteral, intracisternal, intrasternal, intravaginal, intraperitoneal, topical, transdermal, buccal, or as an oral or nasal spray. Accordingly, the pharmaceutically effective compositions may also include pharmaceutically acceptable additives, carriers or excipients. Such pharmaceutical compositions may also include the active ingredients formulated together with one or more non-toxic, pharmaceutically acceptable carriers specially formulated for oral administration in solid or liquid form, for parenteral injection or for rectal administration according to standard methods known in the art.

The term “parenteral” administration refers to modes of administration which include intravenous, intramuscular, intraperitoneal, intracisternal, intrasternal, subcutaneous and intraarticular injection and infusion. Injectable mixtures are known in the art and comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form may be brought about by the inclusion of agents which delay absorption such as aluminum monostearate and gelatin. Injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

In some cases, to prolong the effect of the drug, it is desirable to slow drug absorption from subcutaneous or intramuscular injection. This may be accomplished by using a liquid suspension of crystalline or amorphous material with poor water solubility. The rate of absorption of the drug then depends upon its rate of dissolution which, in turn, may depend upon crystal size and crystalline form. Alternatively, absorption of a parenterally administered drug form may be delayed by dissolving or suspending the drug in an oil vehicle.

To prepare the pharmaceutical compositions of the present invention, an effective amount of the aforementioned agent can be intimately admixed with a pharmaceutically acceptable carrier according to conventional pharmaceutical compounding techniques to produce a dose. A carrier may take a wide variety of forms depending on the form of preparation desired for administration, e.g., oral or parenteral.

Actual dosage levels of active ingredients in the pharmaceutical compositions of this invention may be varied so as to obtain amounts of the active agents which are effective to achieve the desired therapeutic response for a particular patient, compositions and mode of administration. The selected dosage level will depend upon the activity of the active agents, the route of administration, the severity of the condition being treated and the condition and prior medical history of the patient being treated. However, it is within the skill of the art to start doses of the agents at levels lower than required to achieve the desired therapeutic effect and to gradually increase the dosage until the desired effect is achieved.

Compositions according to the present invention may also be administered in combination with other agents to enhance the biological activity of such agents. Such agents may include any one or more of the standard anti-HIV agents which are known in the art, including, but not limited to, azidothymidine (AZT), dideoxycytidine (ddC), and dideoxyinosine (ddI). Additional agents which have shown anti-HIV effects and may be combined with compositions in accordance to the invention include, for example, raltegravir, maraviroc, bestatin, human chorionic gonadotropin (hCG), levamisole, estrogen, efavirenz, etravirine, indomethacin, emtricitabine, tenofovir disoproxil fumarate, amprenavir, tipranavir, indinavir, ritonavir, darunavir, enfuvirtide, and gramicidin.

As mentioned above, the studies reported herein support the use of activators of apoptosis for the ablation of pathogenic HIV-infected cells that destroy the immune system. Thus, one or more of the above-described therapeutic agents can be administered in combination with an apoptosis inducer or activator.

Examples include cytotoxic antibiotics, such as anthracyclins (doxorubicin, idarubicin, and mitoxantrone), those targeting the endoplasmic reticulum (ER) (thapsigargin, tunicamycin, brefeldin), those targeting mitochondria (arsenite, betulinic acid, C2 ceramide) or those targeting DNA (Hoechst 33343, camptothecin, etoposide, mitomycin C). Additional examples include chemotherapeutic agents, antimitotic agents, DNA intercalating agents, taxane, gemcitabine, alkylating agents, platin based components such as cisplatinum and preferably oxaliplatinum and a TLR-3 ligand. Other examples include Actinomycin D, Camptothecinm, Cycloheximide, Dexamethasone, Etoposide, Staurosporine, Colchicine, Doxorubicin.HCl, Genistein, Genistein, Okadaic acid, Phorbol-12-myristate13-acetate (PMA), Anisomycin, Tamoxifen citrate, Betulinic acid, Thapsigargin, Rosiglitazone, Brefeldin A, lonomycin, Rapamycin, Tyrphostin, and Mitomycin C. See, e.g., Casares et al. J Exp Med. 202, 1691-701 (2005) and US Application NO. 20100016235.

The above-described active agents or a composition containing the agents can be used to treat or inhibit an HIV infection. Accordingly, the invention also features methods for treating in a subject has, or is suspected of having, an HIV infection.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model (such as non-human primates). A subject to be treated can be identified by standard diagnosing techniques for the disorder.

“Treating” or “treatment” refers to administration of a compound or agent to a subject, who has a disorder (such as an HIV infection), with the purpose to cure, alleviate, relieve, remedy, delay the onset of, prevent, or ameliorate the disorder, the symptom of the disorder, the disease state secondary to the disorder, or the predisposition toward the disorder. The terms “prevent,” “preventing,” “prevention,” “prophylactic treatment” and the like refer to reducing the probability of developing a disorder or condition in a subject, who does not have, but is at risk of or susceptible to developing a disorder or condition. A “therapeutically effective amount” refers to the amount of an agent sufficient to effect beneficial or desired results. A therapeutically effective amount can be administered in one or more administrations, applications or dosages and is not intended to be limited to a particular formulation or administration route.

The agent can be administered in vivo or ex vivo, alone or co-administered in conjunction with other drugs or therapy. As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

In an in vivo approach, the above-described agent, e.g., CPX or DEF, is administered to a subject. Generally, the agent is suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily. In an ex vivo approach, a subject's blood can be withdrawn and treated with the above-mentioned agent and then the blood thus-treated is given back to the subject.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100 mg/kg. Variations in the needed dosage are to be expected in view of the variety of agents available and the different efficiencies of various routes of administration. Variations in these dosage levels can be adjusted using standard empirical routines for optimization as is well understood in the art. Encapsulation of the agent in a suitable delivery vehicle (e.g., polymeric microparticles or implantable devices) may increase the efficiency of delivery.

Unlike microbicides and antiretroviral therapy (ART), which require constant adherence, as a prophylaxis the active agent described herein can be administered once or a few times in a short course, soon after virus exposure or during the early phases of the infection, in order to purge a substantial fraction, if not all, of virus-harboring cells from the infected individuals. A significant reduction of viral burden in HIV-infected individuals should have a significant impact in preventing or delaying disease progression of these individuals, as well as reducing virus transmission to the community. The above-described compounds may also be applied as a therapeutic agent, in conjunction with or after successful ART to eradicate most, if not all, virus-infected cells that remain. Hence, the use of therapeutic agent has the potential to shorten, or perhaps eliminate, ART, which is currently considered to be lifelong.

In one example, the above-described agent, e.g., CPX or DEF, may be administered fpr about 4 weeks or longer. The capacity of the agent to purge a substantial fraction of virus-harboring cells from the infected individuals has a considerable impact in delaying disease progression and decreasing the duration of ART in these individuals, as well as reducing virus transmission to the community.

The antifungal agent CPX and the medicinal chelator DEF, inhibit retroviral gene expression with concomitant activation of the intrinsic pathway of apoptosis, resulting in the preferential ablation of infected cells. In isolate-infected cultures of primary cells, the drugs produced lasting off-medicine remission, assessed as absent rebound of replication-competent virus and long-term failure of resurgent retroviral expression. No damage to the uninfected cells of epithelial tissues was detected. These data suggest that medicinal activation of apoptosis in pathogenic infected cells is a viable antiviral strategy, for which the term “therapeutic reclamation of apoptotic proficiency” (TRAP) is used herein.

### Immunogenic Composition

The reagents described above can be used in a vaccine formulation to immunize an animal. Thus, this invention also provides an immunogenic or antigenic composition (e.g., a vaccine) that contains a pharmaceutically acceptable carrier and an effective amount of (i) one or more cells that have been infected with HIV-1 and (ii) an iron-chelating hydroxypyridinone described above. The carriers used in the composition can be selected on the basis of the mode and route of administration, and standard pharmaceutical practice.

The term “immunogenic” refers to a capability of producing an immune response in a host animal against a viral (e.g., HIV) antigen or antigens. This immune response forms the basis of the protective immunity elicited by a vaccine against a specific infectious organism. “Immune response” refers to a response elicited in an animal, which may refer to cellular immunity, humoral immunity or both.

The immunogenic or antigenic composition can contain an adjuvant. Examples of an adjuvant include a cholera toxin, Escherichia coli heat-labile enterotoxin, liposome, unmethylated DNA (CpG) or any other innate immune-stimulating complex. Various adjuvants that can be used to further increase the immunological response depend on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum.

A vaccine formulation may be administered to a subject per se or in the form of a pharmaceutical or therapeutic composition. Pharmaceutical compositions containing an antigenic agent of the invention and an adjuvant may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the antigens of the invention into preparations which can be used pharmaceutically. Proper formulation is dependent upon the route of administration chosen.

For injection, vaccine preparations may be formulated in aqueous solutions, preferably in physiologically compatible buffers such as Hanks' solution, Ringer's solution, phosphate buffered saline, or any other physiological saline buffer. The solution may contain formulatory agents such as suspending, stabilizing and/or dispersing agents.

### Viral Versus Therapeutic Control Over Programmed Cell Death

Apoptosis is a typical cellular defense against viral attack. Numerous viruses neutralize this innate initial defense, including HIV-1. Rapid execution of apoptosis occurs upon entry of HIV-1 into “resting” CD4⁺ T cells, before the completion of reverse transcription, but only a fraction of target T cells is able to mount this innate antiviral response. HIV-1 entry leads to expression of viral gene products, many of which interact with cellular components and modulate cellular activities including apoptosis. As shown herein, CPX and DEF allow completion of the innate antiviral suicide response by promoting TRAP, thereby overcoming the retrovirally triggered resistance to apoptosis.

Protein Hydroxylation, eIF5A, and Apoptosis

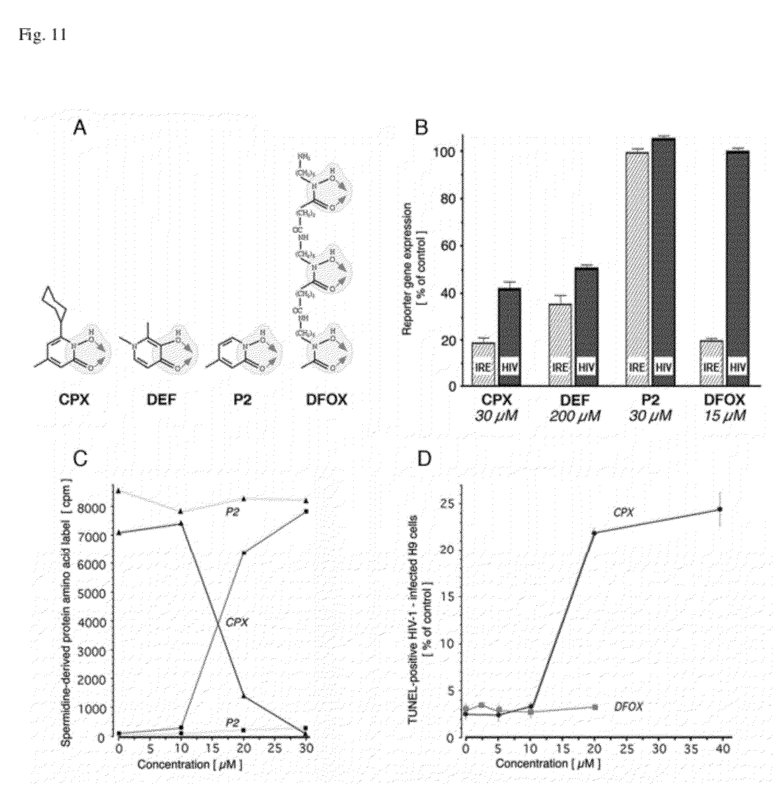

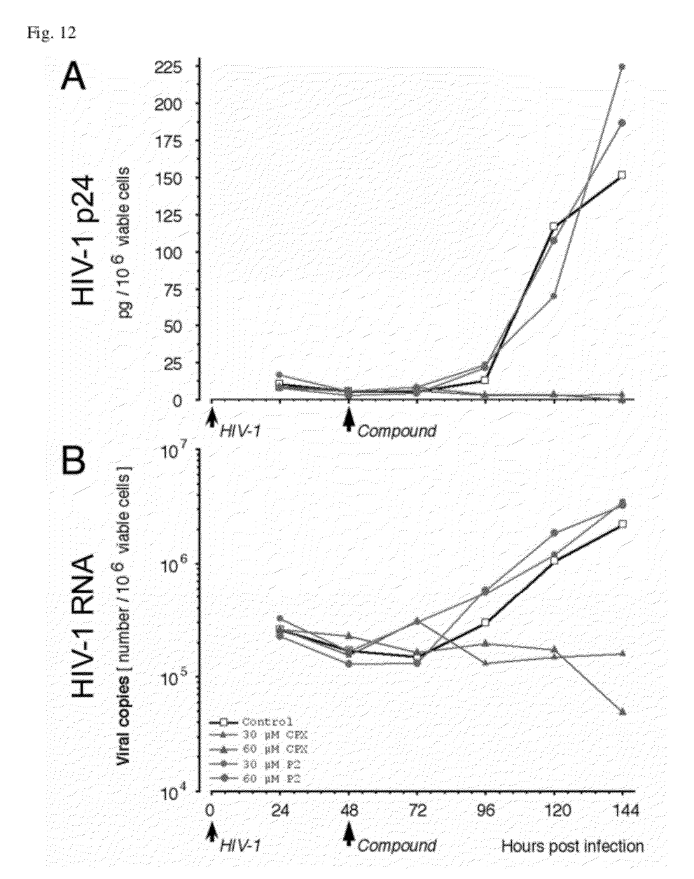

CPX and DEF are both metal chelators, but comparison with DFOX and Agent P2 indicates that metal binding is not sufficient for their antiviral and pro-apoptotic activities (FIGS. 11 and 12). CPX and DEF, but not Agent P2 or DFOX at clinically relevant concentrations, block non-heme iron oxygenases such as deoxyhypusine hydroxylase (DOHH), the enzyme required for the final step in the formation of hypusine. This posttranslationally modified amino acid has been found exclusively in eIF5A, which in cells exists exclusively in the hydroxylated, hypusine-containing form. eIF5A is expressed in lymph nodes during acute HIV-1 infection, is overexpressed in lymphocytes of HIV-1 infected patients, and has been implicated as a cofactor for HIV-1 replication as well as in apoptosis triggered through the intrinsic mitochondrial pathway. Hypusine is essential for eIF5A function in HIV-1 infection and apoptosis. Specifically, pharmacologically or genetically induced inhibition of hypusine formation in culture both activates apoptosis in susceptible cells and inhibits infection by human or feline immunodeficiency virus. Although CPX and DEF could affect other non-heme metalloenzymes with an active site architecture or catalytic metal coordination similar to DOHH, the data disclosed herein indicate that DOHH and the posttranslational modification of eIF5A are likely targets for both drugs. Thus, the inhibition of cellular DOHH by CPX or DEF correlates with each agent's antiviral and pro-apoptotic profile and the dose-response relation for DOHH inhibition by CPX minors the drug's pro-apoptotic activity (FIGS. 11B and C and FIG. 12).

### A Mechanism for Trap and its Consequences

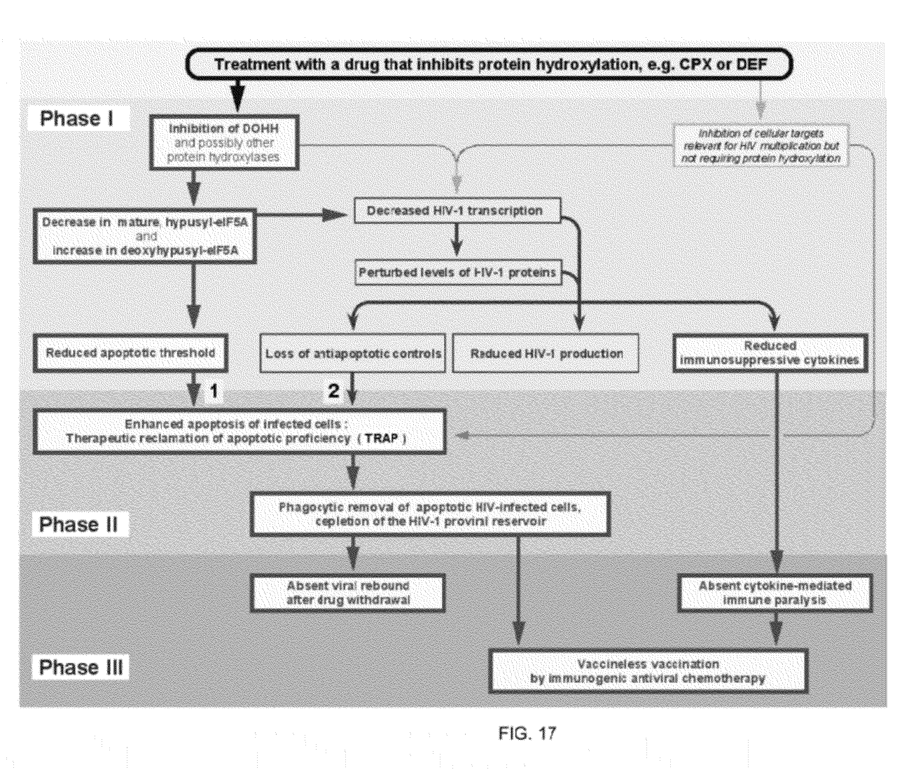

Based on the data presented here, in one embodiment, the invention provides a three-phase model as illustrated in FIG. 17. In Phase I, HIV-1 gene expression is disrupted and production of infective virions and other viral products inhibited, at least in part due to suppression of hypusine formation in eIF5A. These events combine to limit HIV-1 control over the survival of infected cells, leading to TRAP in Phase II. The ablation of infected cells diminishes HIV-1 production sites to the point of eradication, evidenced by the lack of HIV-1 rebound after drug withdrawal in Phase III.

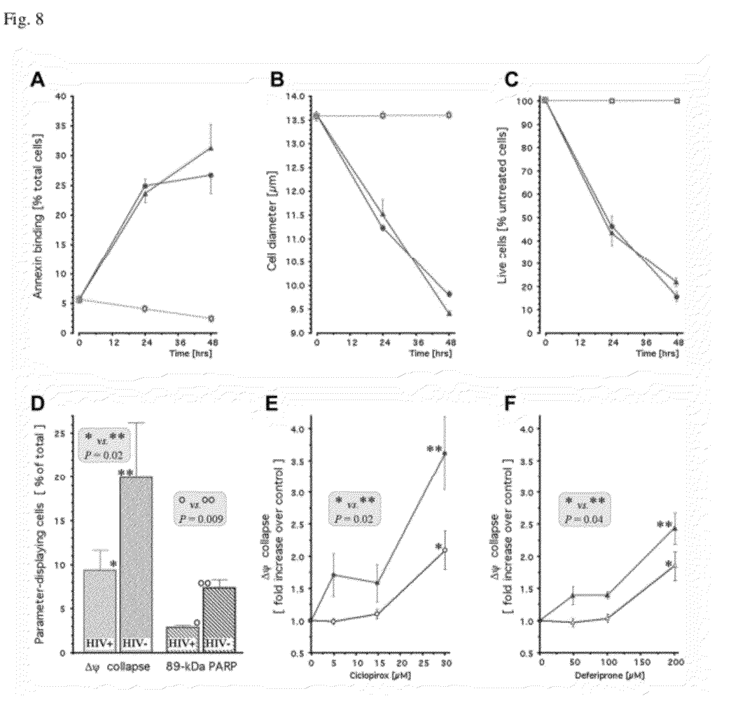

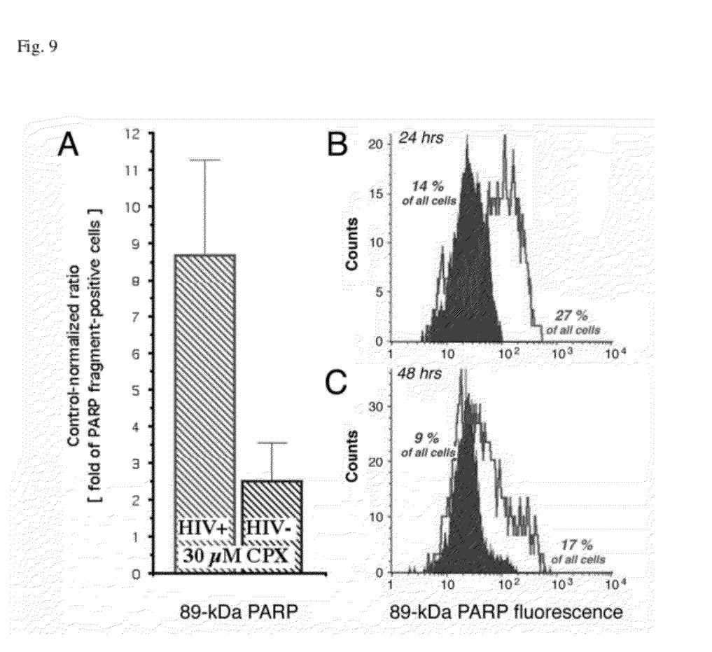

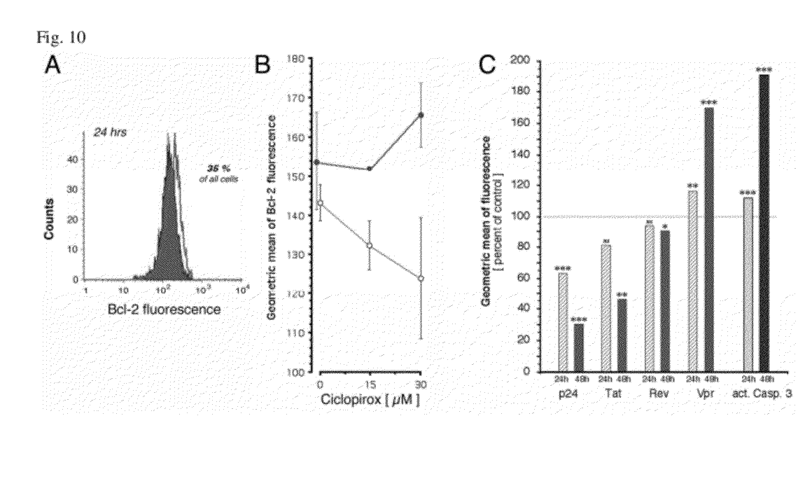

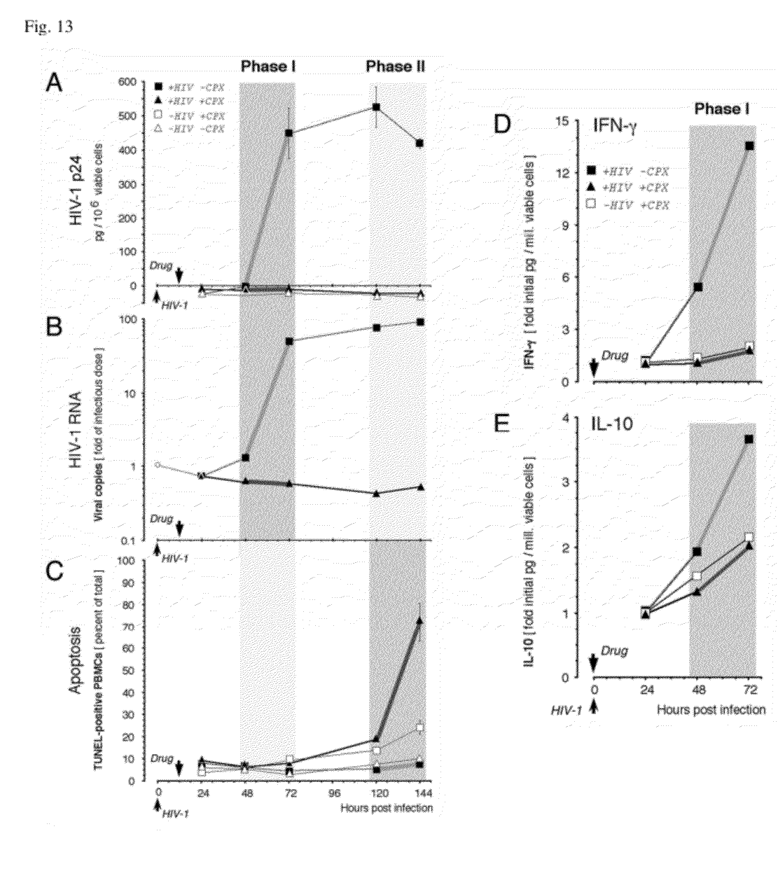

In Phase I, CPX and DEF inhibit DOHH causing depletion of mature eIF5A and accumulation of its non-hydroxylated precursor. This lowers the apoptotic threshold, consistent with the proapoptotic effect of hypusine inhibition. It also blocks HIV-1 gene expression (FIG. 11B), restricting both productive infection (FIG. 12; FIGS. 13A and B) and antiapoptotic effectors like Tat (FIG. 10C). Paradoxically, Vpr increases, and consistent with its apoptogenic activity causes a synchronous rise of active caspase-3 (FIG. 10C). Vpr can originate from incoming virions and is able to extend its half-life independent of synthesis by manipulating the ubiquitin/proteasome pathway. This auto-regulatory activity of Vpr, essential for preintegration transcription and initiation of infection, is restrained by the subsequent expression of HIV proteins like Vif. The drug-mediated suppression of HIV-1 gene expression (FIG. 11B) may result in loss of such feedback control over Vpr. The net effect is an enhancement of the drugs' pro-apoptotic effect particularly in HIV-1 infected cells (FIGS. 8E and F, FIGS. 9 and 13C). As shown in the model, the drugs turn the apoptogenic activity of Vpr against HIV-1 and repurpose this retroviral molecule for the killing of cells in which it occurs.

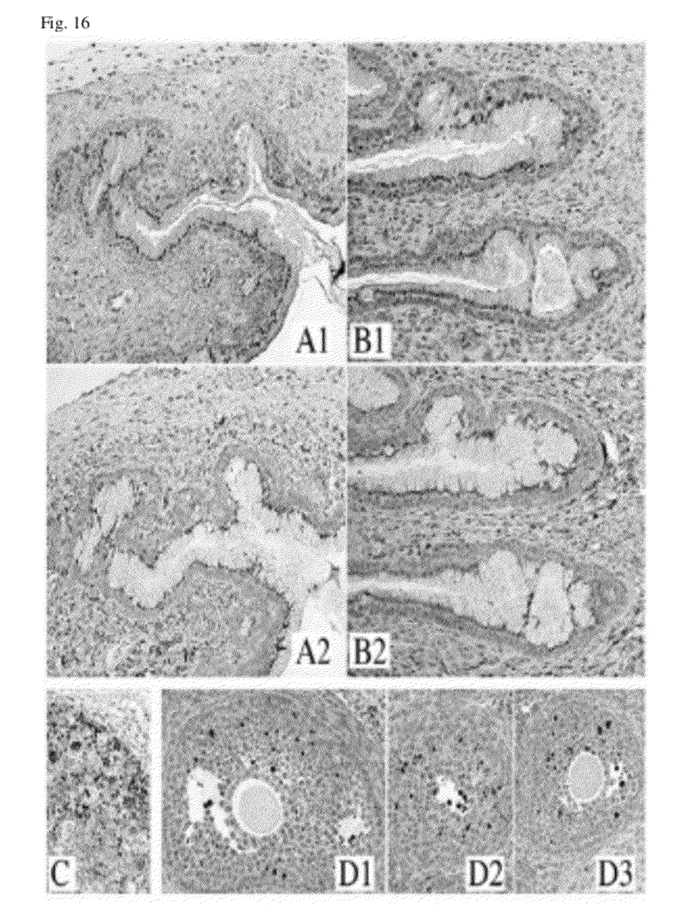

In Phase II, the drug-induced disruption of eIF5A hydroxylation and HIV-1 gene expression convert the retroviral blockade of apoptosis (FIG. 8D) into extensive and preferential death of infected cells (FIG. 13C). The Vpr rise coincides with the increase in active caspase-3 (FIG. 10C), and the accumulation of non-hydroxylated eIF5A directly correlates with apoptosis (FIGS. 11C and D). In the model, the drug-mediated disruption of viral regulatory mechanisms forces HIV-infected cells over the drug-lowered apoptotic threshold, resulting in depletion of the proviral reservoir and the purging of viral sanctuaries. Uninfected cells need to contend solely with a lowered apoptotic threshold. Consequently, in tissue culture the majority of uninfected cells exposed to CPX or DEF survives, in contrast to infected cells (FIGS. 8A-C; 13C, and 15). In vivo, cell populations survive intact even when exposed to drug concentrations that are, as in the case of CPX, orders of magnitude above those causing the death of infected cells (FIG. 16). By blocking retroviral gene expression (FIG. 11B), the drugs also quench the virally induced production of immune paralysis-promoting host molecules, exemplified by Tat-induced IL-10 (FIG. 13D). IL-10 disrupts virus-specific T cell responses and causes persistence of viral infection.

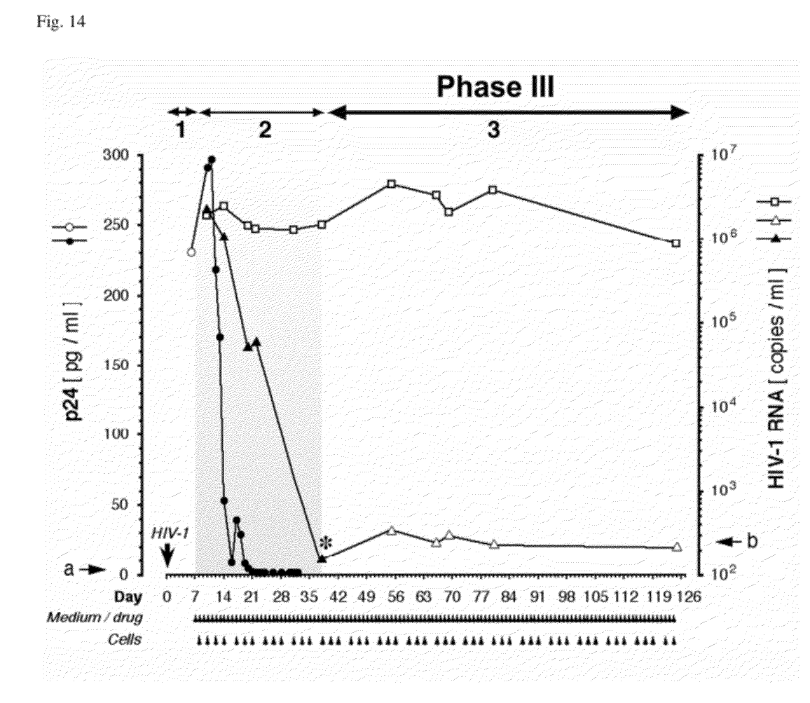

In Phase III, the consequence of suppressed infection in Phase I and of activated apoptosis in Phase II emerges as functional termination of established virion production, evidenced by the absence of post-treatment rebound (FIG. 14). This dual activity imitates the adaptive immune response in emulating two of its major effects, namely the blockade of acute infection (by neutralizing antibodies) and the ablation of virally infected cells (by cytotoxic lymphocytes). In addition to this immunomimetic activity, evident in culture (FIGS. 12-14), such drugs display immunogenic activity in vivo, according to the following model:

The blockade of HIV-1 gene expression (FIG. 11B) reduces immune paralysis-mediating retroviral and cellular products, e.g. Tat (FIG. 10C) and IL-10 (FIG. 13E), resulting in protection of antigen presentation by dendritic cells while the retroviral suppression of defensive host cell apoptosis is reversed (FIGS. 9 and 13C). This constellation terminates the retroviral evasion of immunogenic cell death: The infected cells, rendered apoptotic in situ by pharmacological means, serve as endogenous vehicles that deliver retroviral immunogens to the pharmacologically sustained, or de-paralyzed, immune system.

HIV-infected PBMCs that are rendered apoptotic ex vivo and re-introduced into a host with a functionally unimpeded immune system, induce HIV-1 specific cellular and humoral responses that effectively protect against challenge with live infected cells. Based on the above-discussed model, small molecules like CPX or DEF render HIV-infected PBMCs apoptotic in vivo, obviating the need for ex vivo manipulations, and deliver them as non-disruptive immunogenic input into the adaptive immune system. This “vaccineless vaccination” results in endogenous suppression of HIV-1 infection. A small trial determining the virological response to DEF in HIV-infected volunteers revealed persistent off-medicine antiretroviral activity even eight weeks after drug withdrawal, consistent with small molecule-initiated and prolonged endogenous suppression.

### Clinical and Drug Development Implications

The medicines investigated in the examples below as antiretroviral pioneer drugs are widely used and considered safe for their approved human applications, as well as nontoxic in experimental animals. DEF achieves plasma levels of up to 350 μM after oral administration to mostly pediatric patients (Andrus et al. (1998) Biochem Pharmacol 55: 1807-1818), in excess of the concentration tested here (FIGS. 8, 11, and 15). As a chronically administered medication of thalassemic children, DEF does not interfere with their physical development, preserves the function of particularly sensitive cells in several tissues, and even reverses organ failure (Pennell et al. (2006) Blood 107: 3738-3744 and Farmaki et al. (2010) Br J Haematol 148: 466-475). CPX is used in gynecological and dermatological preparations that contain from 28.8 mM to 230.2 mM of CPX. The gynecological preparations, provided as ovules, creams, foams, or lavages, are routinely used for up to 14 days to treat vaginal candidiasis, with miniscule systemic absorption (Coppi et al. (1993) J Chemother 5: 302-306). The FDA-approved dermatological preparation is well tolerated when applied periungually for 48 weeks (FDA-DODACNDA21-022 (1999) Scientific and Open Session Files. In: Committee DaODA, editor. Silver Spring, Mass.; USA: Food and Drug Administration, Center for Drug Evaluation and Research.). The most recent review of the literature did not disclose clinically relevant adverse effects suggestive of local apoptotic damage to human tissues caused by any commercially available, vaginally or cutaneously applied antifungal formulations of CPX (Subissi et al. (2010) Drugs 70: 2133-2152). This is consistent with the result on topical administration in mice (FIG. 16). Also in mice, systemic administration of CPX (25 mg/kg) by daily oral gavages failed to cause ill effects (Zhou et al. (2010) Int J Cancer 127: 2467-2477).

In conclusion, the data indicate that CPX and DEF are prototypes for a novel class of drugs that employ TRAP to kill virally infected cells while blocking infection. This mode of action mitigates the threat of immune system paralysis and the requirement for continuous medication that drives viral resistance. Selective optimization of their antiretroviral and pro-apoptotic side activities can be guided by established steric parameters and structure-activity relations (FIG. 11).

### EXAMPLES

### Materials and Methods

The following materials and methods apply to all examples, unless specifically noted otherwise.

CPX, DEF, and DFOX

CPX, as its mono-ethanolammonium salt (‘ciclopirox olamine’), was obtained from Sigma Chemical Co. (St Louis, Mo.) dissolved in sterile, trace metal-free Earle's Solution (Sigma Chemical Co.). 20 mM stock solutions were maintained at 4° C., used for four weeks, and then discarded. In solutions and buffers containing trace metals and phosphate, CPX forms a faint precipitate that renders their use as stock unreliable. Stock solutions were not frozen, since CPX tends to precipitate upon thawing. For mouse studies, 1% Batrafen Vaginalcrème™, an oil-in-water preparation containing 28.8 mM total and 0.6 mM bioavailable CPX, was obtained from Sanofi-Aventis (Frankfurt, Germany). Drug-grade DEF was provided by Apotex (Toronto, Canada) and DFOX was purchased from Sigma Chemical Co. Stock solutions (20 mM and 2 mM, respectively) were prepared and handled as above. CPX and DEF were used at 30 μM and 200 μM, respectively, except where otherwise specified.

### Synthesis of Agent P2 (1-hydroxy-4-methylpyridin-2[1H]-one)

4-picoline N-oxide (1.14 g, 10.43 mmol) in tetrahydrofuran (54 ml, distilled from sodium/benzophenone under N₂) was cooled to −78° C. in a dry ice-acetone bath. n-Butyllithium (1.6 M in hexanes, 13.0 ml, 20.8 mmol) was added, the red-brown mixture stirred for 1 hr under nitrogen and then oxygen-bubbled for 30 min. Brought to room temperature, water (30 ml) was added, the mixture acidified to pH 2 with hydrochloric acid and extracted with chloroform (8×55 ml). The extracts were dried with Na₂SO₄, filtered, and the filtrate evaporated under reduced pressure. A yellow-brown residue was purified by chromatography (silica gel, ether) yielding 1-hydroxy-4-methylpyridin-2[1H]-one by CHN analysis and the following criteria: 1H NMR (300 MHz, CDCl₃, δ): 10.18 (1H, br, OH); 7.63 (1H, d, J=7 Hz, aromatic); 6.49 (1H, d, J=2 Hz, aromatic); 6.15 (1H, dd, J=7 Hz, 2 Hz, aromatic); 2.22 (3H, s, CH₃). MS (EI): m/z 125 [M⁺.].

Antibodies

Rabbit NIH-353 antibody was raised against mature human eIF5A (Cracchiolo et al. Gynecol Oncol 2004, 94:217-222). Antibody against DOHH was generated by M. H. Park. Anti-eIF5A-1 monoclonal antibody (BD) was purchased from BD Biosciences. The anti-FLAG monoclonal antibody M2 and anti-actin antibody were purchased from Sigma.

H9 and H9-HIV Cell Lines

Uninfected and uniformly HIV-1 (HTLV-IIIB) infected H9 cells, obtained from the NIH AIDS Research and References Reagent Program, were cultured at 37° C. in a humidified atmosphere (5% CO₂, 95% humidity) using RPMI 1640 medium supplemented with 2 mM L-glutamine, 100 μg/ml streptomycin, 100 μg/ml streptomycin and 20% fetal calf serum.

293T and COS7 Cells

The cells were grown in DME medium (Sigma) and Jurkat cells in RPMI medium (Sigma), both supplemented with penicillin, streptomycin and 8% FBS. Quantitation of apoptosis and viability was performed with a BD FACSCalibur™ system using Annexin V-PE Apoptosis Detection Kit I (BD Biosciences, San Jose Calif.).

Plasmids

pSP-luc+ and pSP-rluc were purchased from Promega, Madison. The HIV-1 molecular clone pMRev(−), and the Rev expression vector, plasmid pCMV-Rev, were obtained from the NIH AIDS Research and Reference Reagent Program. FLAG-tagged Rev and eIF5A expression vectors were made by sub-cloning Rev and eIF5A sequences respectively, into the pcDNA3.1FLAG vector. pBSII-HIV+80-340 was constructed by subcloning PCR-amplified HIV-1 sequence (+80-340) from pNL4-3-LucE- (Chen et al. J Virol 1994, 68:654-660) into the pBSIIKS+ Bluescript vector. pNL4-3-LucE- truncations were generated by deleting sequences using suitable restriction enzymes. Truncation III was made by deleting sequence from nt 1506 to 5784 using SpeI and SalI enzymes. Similarly, truncations IV (nt 5784 to 8464) and V (nt 712 to 8464) were made with SalI and BamHI and with BssHII and BamHI, respectively. The plasmid pGL2TAR was obtained from Dr. David Price and contains most of the HIV-1 LTR (from KpnI to HindIII). To generate construct VI, the HindIII to PflM1 sequence from pGL2TAR was eplaced by the HindIII to XhoI sequence from construct V. Construct VII (pLTR-FF) was made by ubstituting the sequence between the ClaI and BsgI sites of pGL2TAR with the ClaI to BsgI fragment from construct VI.

Transfection and Luciferase Assays

Plasmids were introduced into 2×10⁵ 293T cells by transfection using Lipofectamine 2000 (Invitrogen) according to the manufacturer's instructions. Compounds (such as CPX or DEF) were added simultaneously. Cells were harvested at 12 hr post-transfection, washed with PBS, lysed in 0.15 ml of 1× passive lysis buffer (Promega), and assayed for luciferase activity using the Promega dual luciferase reporter system according to the manufacturer's instructions. Jurkat cells (1×106 cells) were transfected using FuGENE 6 (Roche) according to the manufacturer's instructions and assayed in a similar fashion after pelleting.

Preparation of Nuclear and Cytoplasmic RNA

293T cells (2×106 cells) were seeded in 10-cmdiameter plates and transfected 20 hr later by using Transfectene (Bio-Rad) and treated with compounds. Cells were harvested at 15 hr post-transfection and suspended in a low salt buffer (10 mM Tris.HCl pH 7.4, 10 mM NaCl, 1.5 mM MgCl2, and 0.5% NP-40). Cells were vortexed for 10 sec and incubated on ice for 10 min. Cell extracts were centrifuged at 500×g for 3 min, followed by cytoplasmic and nuclear RNA isolation from the supernatant and the pellet, respectively, using Trizol (Invitrogen) according to the manufacturer's instructions.

RNase Protection Assay (RPA)

RPA was performed with 10 μg of cytoplasmic RNA and 5 μg of nuclear RNA, using the RPAIII kit from Ambion (Austin, Tex.) according to the manufacturer's instructions. Synthesis of radiolabeled RNA and protection assays were performed as described in Young et al. Mol Cell Biol 2003, 23:6373-6384. To generate antisense RNA probe against the HIV-1 major splice site, firefly luciferase and Renilla luciferase pBSII-KS+HIV (+80-341), pSP-luc and pSP-rluc were linearized with HindIII, XbaI and BsaI, respectively. The resulting probes were 309, 390 and 245 nt long, respectively. The antisense HIV-1 leader RNA probe complementary to nt+83 to −117 of the LTR was generated by subcloning between the XbaI and HindIII sites of the pcDNA3.1 vector. Antisense probe corresponding to the N terminus of eIF5A was generated by subcloning 250 nt of its cDNA sequence into pcDNA3.1.

Immunoprecipitation and Immunoblotting

Immunoprecipitation and immunoblotting experiments were carried out as described previously in Hoque et al. Mol Cell Biol 2003, 23:1688-1702.

RNA Interference

A pool of four siRNAs targeting eIF5A-1 mRNA (ON-TARGETplus SMARTpool®), sequence-specific siRNA against DOHH, and control siRNA were purchased from Dharmacon Inc. Cells were transfected with 50 nM siRNA using HiPerFect transfection reagent (Qiagen) according to the manufacturer's instructions. The effectiveness of siRNA against specific targets was determined by RPA and immunoblotting.

Uninfected PBMCs

Using an institutional review board (IRB)-approved protocol, PBMCs were isolated from the blood of healthy donors and stimulated overnight with phytohemagglutinin (PHA) and human IL-2. Stimulated cells were pelleted and resuspended for culture at a final concentration of 0.5×10⁶ cells/ml in PHA-free RPMI 1640 medium containing 10% fetal calf serum (v/v), 100 units/ml penicillin G, 100 μg/ml streptomycin, 2 mM glutamate, and 3.5 ng/ml human IL-2 (Medium B). Cultures were incubated at 37° C., 5% CO₂, and 95% humidity.

Infectious Virus Stock

Using an IRB-approved protocol, two donors were recruited to generate clinical viral isolates. One (#990,135) was highly immunocompromised despite on-going combination antiretroviral therapy (cART) (CD4 count <5%; HIV RNA in plasma at log₁₀ 5.5 copies/ml). The second (#990,010) was moderately to severely immunocompromised on cART (CD4 count 14-26%; HIV RNA in plasma at log₁₀ 3.8-5.0 copies/ml). For infection, 5×10⁶ uninfected stimulated PBMCs were co-cultured with 10×10⁶ PBMCs from one of the HIV-infected donors in Medium B. On day 3, half of the supernatant was removed and replenished with an equal volume of fresh Medium B. On day 7, the medium was likewise replenished and 7.5×10⁶ stimulated uninfected PBMCs were added. On days 10, 17, and 24, half of the supernatant was replenished. On days 14, 21, and 28, stimulated uninfected PBMCs were added. Cells were harvested when p24 reached 250 pg/ml, cryopreserved in freezing medium (90% fetal calf serum, 10% dimethyl sulfoxide), and stored in liquid nitrogen as infected PBMC stock. Cell-free supernatants were stored at −80° C.

Acute PBMC Infection Model

Uninfected PBMCs were co-incubated with infected PBMC stock in 24-well microplates at 2 ml/well Medium B, at a 10:1 ratio of uninfected-to-infected cells, and a total cell number of 1×10⁶. Cultures were maintained and assayed for 6 consecutive days. CPX or Agent P2 was added at the time of inoculation or 12 hr later. Leaving the cell layer undisturbed, half the medium in each well was replenished every day, with concurrent adjustment of compound concentration. On each day during the 6-day experiments, a set of wells was harvested: the cells were processed for determination of viability and apoptosis, and the cell-free supernatants were stored at −80° C. for p24 and viral RNA measurements. Experiments were performed at least in duplicate and repeated at least twice.

Persistent PBMC Infection Model

Uninfected (5×10⁶ cells) and infected PBMCs (0.5×10⁶ cells) were co-incubated in a 25 ml culture flask at a final concentration of 0.22×10⁶ cells/ml. Cultures were allowed to establish productive infection, defined by medium p24 at or above 250 pg/ml, and CPX was added. Cultures were replenished with Medium B and freshly isolated uninfected PBMCs on alternate days. For replacement of Medium B, half of the supernatant was gently exchanged without disturbing the cells, and the drug concentration was adjusted appropriately. For replacement with freshly isolated, stimulated and uninfected PBMCs, half of the cells and supernatant were removed and replaced with 2.5×10⁶ cells in the proper volume of Medium B, with adjustment of the drug concentration. Cell-free supernatants were saved for p24 and viral RNA measurements.

Quantitation of Cell Number, Viability, and Diameter

Viability, diameter, and cell number of PBMCs and H9 cells were measured by computerized image analysis of trypan blue exclusion (VI-CELL™; Beckman Coulter; Fullerton, Calif.). PBMCs were counted with an automated, multi-parameter hematology analyzer (CELL-DYN™; Abbott Laboratories, Abbott Park, Ill.).

Mitochondrial Membrane Potential (ΔΨ) and DNA Fragmentation Assays

The potential across the mitochondrial membrane of live cells was determined flow-cytometrically with the lipophilic cationic fluorochrome JC-1 (5,5′,6,6′-tetrachloro-1,1′,3,3′-tetraethylbenzimidazolcarbocyanine iodide) immediately after sample harvest (BD™ Mitoscreen Kit, BD Biosciences; San Diego, Calif.). DNA fragmentation was quantified flow-cytometrically, using a TUNEL (terminal deoxynucleotide transferase dUTP nick end-labeling) assay (APO-BRDU™; Phoenix Flow Systems; San Diego, Calif.) and a dual-color assay for annexin V binding and 7-AAD exclusion (Annexin V-PE 7-AAD Apoptosis Detection Kit I™; BD Biosciences Pharmingen, San Jose, Calif.). The apoptotic volume decrease of cells was assessed by the reduction of their diameter (VI-CELL™; Beckman Coulter; Fullerton, Calif.).

Quantitation of Intracellular Proteins

Intracellular antigens were determined, as geometric means of fluorescence, by flow cytometric analysis using a BD FACSCalibur™ and its BD FACStation System™ (Becton Dickinson; San Jose, Calif.). Intracellular antigens were stained according to the instrument producer's instructions. Anti-HIV-1 Tat antibody was from Abcam Inc. (Cambridge, Mass.); anti-HIV-1 Vpr from Santa Cruz Biotechnology (Santa Cruz, Calif.); anti-HIV-1 Rev from Advanced Biotechnologies Inc. (Columbia, Md.); anti-HIV-1 p24 (KC57) from Beckman Coulter, Miami, Fla.; monoclonal C92-605 anti-human caspase-3 (active form) from Pharmingen (BD Biosciences, San Diego, Calif.); monoclonal F21-852 anti-human poly (ADP-ribose) polymerase (caspase-cleaved 89-kDa fragment); and monoclonal 6C8 anti-human Bcl-2 from Becton Dickinson (BD Biosciences; San Diego, Calif.).

Gene Expression Assays

293T cells (10⁵ cells/well of 12-well microplates) were seeded and transfected 20 hr later with a firefly luciferase (Luc)-expressing reporter plasmid and corresponding Renilla luciferase (Ren)-expressing reference plasmid using TransFectin™ (Bio-Rad, Hercules, Calif.). The bioavailable intracellular iron concentration was monitored using plasmids obtained from B. Galy and M. W. Hentze (EMBL, Heidelberg). Plasmid pcFIF-Luc contains the mouse ferritin H 5′-UTR with an iron response element (IRE) controlling Luc; pcFIRo-Ren is a similar construct expressing a mutated non-functional IRE fused to Ren. The activity of the HIV-1 promoter was monitored using the pNL4-3-Luc E molecular clone (Chen et al. (1994) J Virol 68: 654-660.), based on the recombinant infectious proviral clone that contains DNA from HIV isolates NY5 (5′ half) and IIIB (3′ half). This plasmid, obtained from D. Baltimore (Caltech, Pasadena), contains the Luc gene in place of 102 nucleotides from nef and 6 nucleotides from env. pCMV-Ren (Promega, Madison, Wis.) was used as reference for pNL4-3-Luc E. Compounds were added at the time of transfection. Cells were harvested 12 hr later, washed with phosphate-buffered saline, and lysed for the dual luciferase assay (Dual Luciferase™ Reporter Assay System; Promega, Madison, Wis.). Luc data were normalized to the Ren internal controls (Hogue et al. (2009) Retrovirology 6: 90.).

Quantitation of p24, Viral Copy Number, and Cytokines in Media

p24 core antigen in the supernatant was quantified by ELISA (Retrotek HIV-1 p24™; ZeptoMetrix Corp.; Buffalo, N.Y.). HIV-1 RNA copy number in the supernatant was determined with a PCR-based and FDA-approved assay (Amplicor HIV-1 Monitor™; Roche Diagnostics Corp.; Indianapolis, Ind.). IFN-γ and IL-10 in the supernatant were determined by cytometric bead array (Human Th1/Th2 Cytokine Kit II™; BD Biosciences Pharmingen, San Jose, Calif.).

Drug Toxicity Measurement in Cell Culture

The human uterine epithelial cell line ECC-1 was cultured in transwell inserts in special, insert-accommodating 24-well plates (Fisher Scientific; Pittsburgh, Pa.) as described in Fahey et al. (2005) Hum Reprod 20: 1439-1446 and Richardson et al. (1995) Biol Reprod 53: 488-498. This establishes an epithelial barrier-forming system of polarized, tight junction-linked human epithelial cells with both apical and basolateral compartments. As an indicator of tight junction formation, trans-epithelial resistance (TER) was measured using an EVOM electrode and Voltohmmeter (World Precision Instruments, Inc., Sarasota, Fla.). Once the seeded ECC-1 reached maximal epithelium-like barrier functions, ascertained by TER ≧1000 ohms/cm², drugs were added to some wells and TER measurements taken on consecutive days. Medium supplemented with the appropriate amount of drug was replenished every day in the apical chamber, and every other day in the basolateral chamber. At least two independent experiments were conducted with a minimum of 4 wells per drug or control.

Mouse Model for Cervicovaginal Toxicity

As described in previously in Cone et al. (2006) BMC Infect Dis 6: 90, 10 week old female CF-1 mice (Harlan; Indianapolis, Ind.) received 2.5 mg medroxyprogesterone acetate (MPA) administered subcutaneously to induce superficial mucification of the vagina, i.e. a surface layer of vital columnar instead of dead cornified cells. Five days after injection, three groups of ten animals each were formed. Animals in Groups A and B received 20 μl 1% Batrafen Vaginalcrème™ containing 28.8 mM total and 0.6 mM bioavailable CPX on four consecutive days. Animals in Group C (controls) received 20 μl phosphate buffered saline on four consecutive days. All animals were then challenged by vaginal inoculation with HSV-2, the animals in Groups A and C receiving high-dose (10 ID₅₀) and those in Group B low-dose (0.1 ID₅₀) inoculum. Vaginal lavages from all animals were obtained three days after inoculation and assayed for viral shedding using an anti-HSV-2 FITC-conjugated mouse monoclonal antibody for direct fluorescence detection of HSV-2 antigen expression (Bartels™ Herpes Simplex Virus Type-Specific Fluorescent Monoclonal Antibody Test; Trinity Biotech, Bray, Ireland) in cell cultures inoculated with the vaginal lavages. Bright green fluorescence of the inoculated wells was read as an HSV-2-positive reaction. In this murine model of vaginal susceptibility to infection in situ, the high-dose inoculum infects on average 87% and the low-dose inoculum 13% of untreated control animals.

To assess the histological effect of this drug regimen on the vaginal mucosa, two test animals pretreated with MPA received 20 μl Batrafen for three consecutive days, two control animals pretreated with MPA received no further treatment, and two additional control animals were untreated. On the third day, 2.5 hours after delivering the third dose of Batrafen Vaginalcrème™ to the test animals, all six animals were humanely sacrificed, the entire genital tract dissected as a single-organ package, fixed in 10% neutral-buffered formalin, paraffin-embedded, sectioned, and stained/counterstained with hematoxylin/eosin. To visualize apoptotic cells in the reproductive tract tissues of untreated and Batrafen-treated mice, sections were stained for the active form of caspase-3 using monoclonal antibody C92-605. This reagent is specific for active caspase-3 and lacks cross-reactivity against pro-caspase-3. Signal generation required the application of a cycled microwave irradiation protocol for antigen retrieval, using a commercially available buffer system (Citra™, BioGenex; San Ramon, Calif.). Immunostaining was optimal at a dilution of 1:125 after overnight incubation. In identically prepared sections of the same tissue blocks that were to be evaluated as negative controls, the anti-human caspase-3 (active form) reagent was omitted. For signal generation, the streptavidin-biotin/horseradish peroxidase complex technique was used, with diaminobenzidine as chromogen and hematoxylin as counterstain. Endogenous peroxidase was blocked in all tissue sections as described in Cracchiolo et al. (2004) Gynecol Oncol 94: 217-222. All slides were examined in a blinded manner by an experienced pathologist specialized in the analysis of human and rodent female genital tract histology.

Data Analysis

Descriptive statistics were generated using Microsoft Excel 2008. Means±SEM were generated for geometric means of fluorescence, at 24 and 48 hr and the ratio of control vs. treated cells was compared with the SEM in a t-test to assess the change relative to the precision of measurement. Time course of cytokines was correlated with the time course of viral parameters. Statistical significance was based on the number of measurements per correlation.

### Example 1

### Inhibition of HIV-1 Gene Expression

CPX and DEF Actions

CPX and DEF both act as potent inhibitors of eIF5A maturation in cells and in vitro. DEF is in clinical use as an orally active medicinal chelator for treatment of transfusional iron overload, and CPX is employed as a topical antifungal. After oral medication, the concentration of DEF in serum can reach and exceed 250 μM (76. Kontoghiorghes et al. Clin Pharmacol Ther 1990, 48:255-261). The topical preparations of CPX, which contain up to 57.5 mM of the agent, achieve levels in excess of 30 μM in skin (Gupta Int J Dermatol 2001, 40:305-310). The results here were obtained at 250 μM DEF and 30 μM CPX, concentrations well within the range of the drugs' clinically relevant levels. At these concentrations, CPX and DEF can reduce bioavailable intracellular iron levels as determined with an iron-sensitive reporter system, but this effect does not correlate with their antiretroviral action.

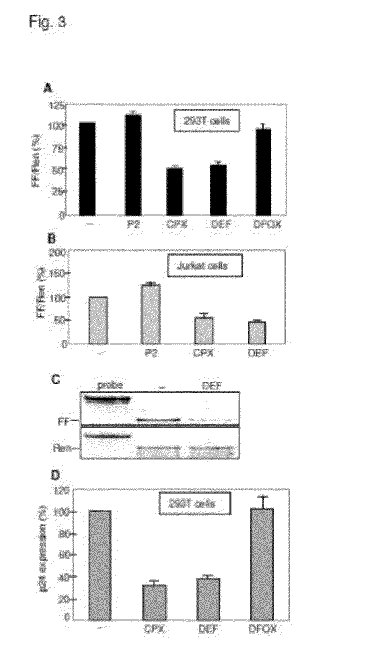

The medicinal chelator DFOX, which also reduced bioavailable intracellular iron levels, did not inhibit gene expression from the HIV molecular clone (FIG. 3) or block HIV-1 replication when used at clinically relevant levels (Lazdins et al. Lancet 1991, 338:1341-1342), consistent with its lack of clinical antiretroviral activity (Salhi et al. J Acquir Immune Defic Syndr Hum Retrovirol 1998, 18:473-478). Several other chelators have been reported to inhibit HIV-1 replication via various possible mechanisms, among them the biologically distinct tridentate drug deferasirox (ICL670) (Nick et al. Curr Med Chem 2003, 10:1065-1076). Agent P2, a bidentate chelation homolog of CPX lacking its hydrophobic cyclohexyl group, displayed little or no activity in the cell-based assays. Thus, the inhibitory action of CPX and DEF on HIV-1 transcription is not merely a consequence of their ability to coordinate and deplete bioavailable iron by bidentate chelation.

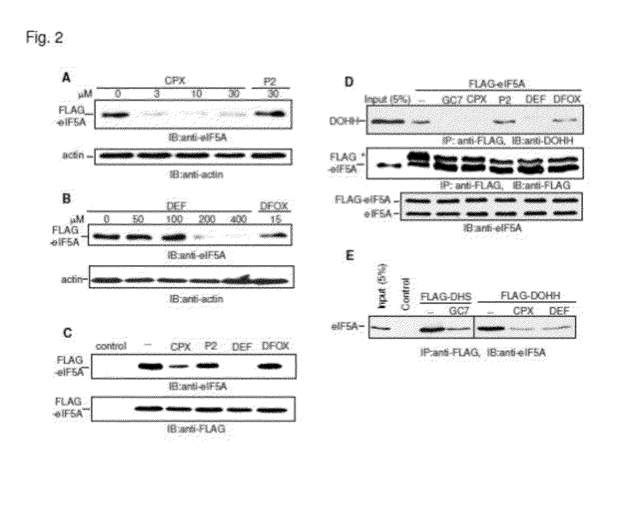

CPX and DEF destabilized the interaction between DOHH and deoxyhypusyl-eIF5A, resulting in a marked decrease in the appearance of newly synthesized mature eIF5A (FIG. 2). The drugs did not prevent eIF5A from forming a complex with DHS, consistent with the accumulation of deoxyhypusyl-eIF5A in the presence of either drug at concentrations that completely blocked DOHH activity (Clement et al. Int J Cancer 2002, 100:491-498). Neither DFOX nor P2 had any effect on the binding of eIF5A to DOHH, in accord with their failure to inhibit the formation of hypusinyl-eIF5A. On the other hand, the DHS inhibitor GC7 blocked formation of lysyl-eIF5A:DHS complexes, causing a marked decrease in the levels of deoxyhypusyl-eIF5A and its complexes with DOHH. These findings, supported by molecular modeling, lead to the proposal that CPX and DEF enter the deoxyhypusine-binding pocket of DOHH, become oriented towards its catalytic iron atom and chelate it. The drug-iron chelate is then released from the apoenzyme, which irreversible collapses into a catalytically inactive molecule incapable of binding substrate. Supporting this mechanism, DEF is known to cause release of peptide-bound iron from several non-heme metalloproteins, among them mono- and diferric transferrin, cyclooxygenase, and lipoxygenase.

Drug Effects on HIV-1 Gene Expression

Assays were carried out to analyze the action of the drugs in a model system consisting of 293T cells transfected with HIV-1 molecular clones. Within 12 hours of their addition, CPX and DEF inhibited gene expression from two different molecular clones, impairing transcription from the viral promoter at the level of initiation. This conclusion is supported by several observations: the inhibition was dependent on the HIV-1 5′-LTR, and no specific effects were detectable on transcription elongation or on downstream mRNA processing, transport or stability. Gene expression from another promoter (the CMV immediate early promoter) and the levels of cellular proteins (actin, eIF5A) were unaffected by CPX and DEF.

eIF5A has also been reported to play post-transcriptional roles in HIV gene expression. Others have identified eIF5A as a cellular cofactor for Rev, leading to the expectation that CPX and DEF would block the export of under-spliced HIV-1 RNAs from the nucleus in a Rev-dependent manner. This prediction was not substantiated in the experiments here, however.

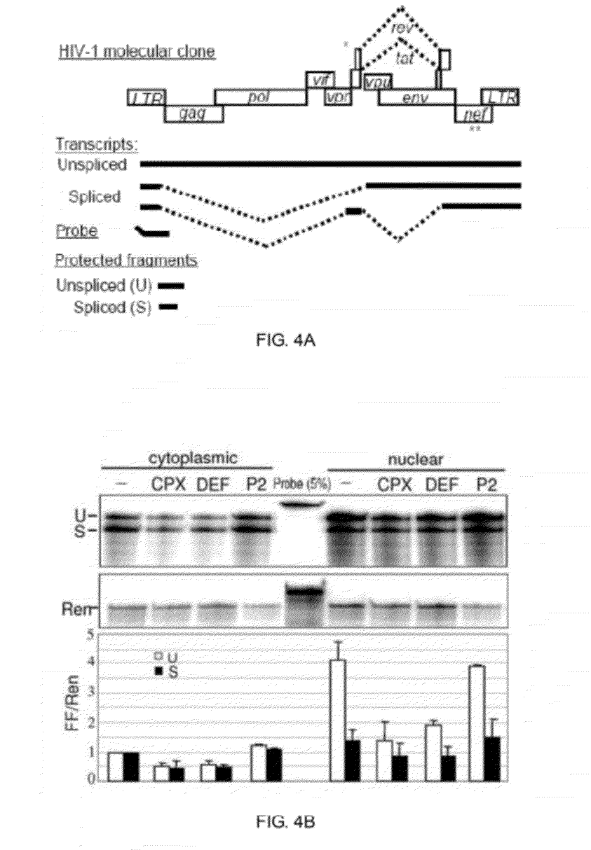

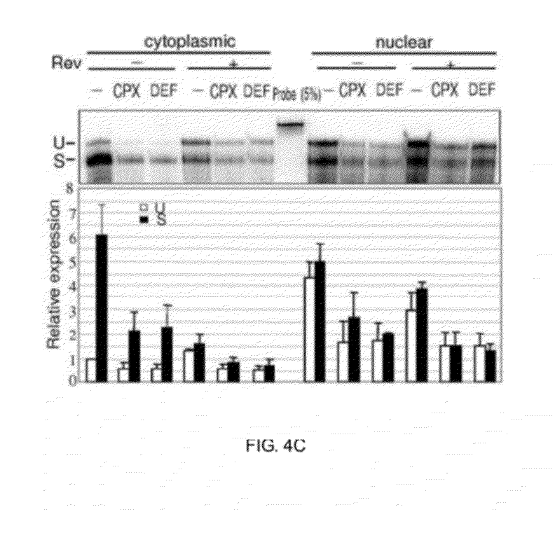

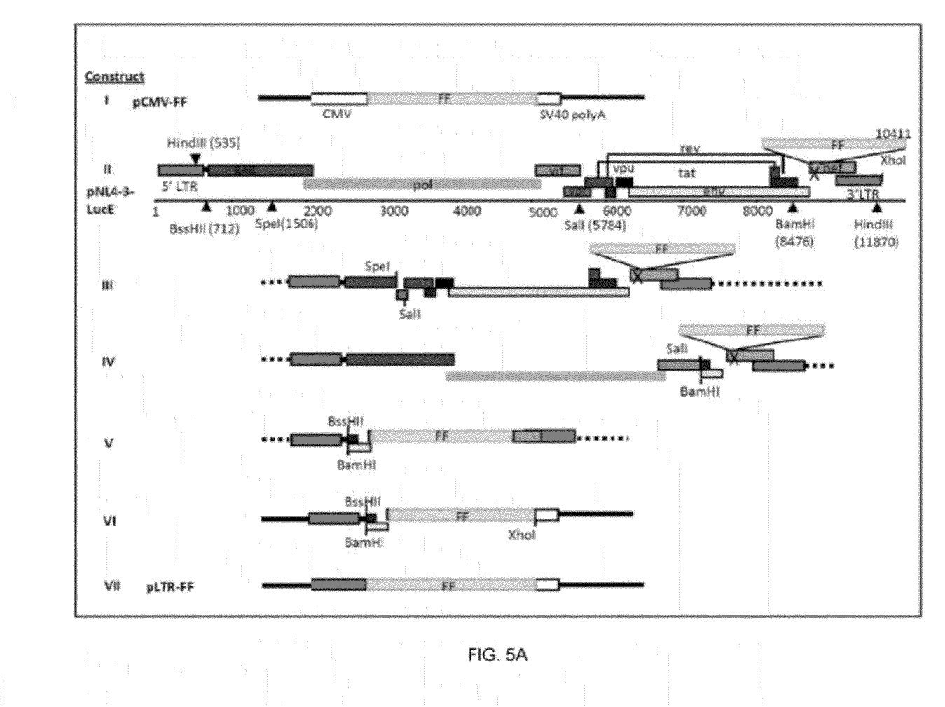

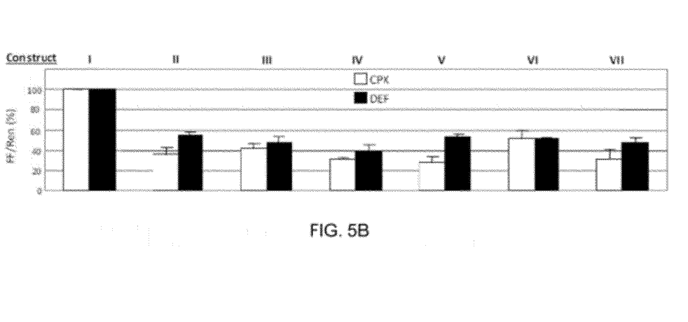

First, the sensitivity of FF expression from pNL4-3-LucE- to the drugs (FIG. 3) implies a Rev-independent action because the FF luciferase gene is inserted into the HIV-1 nef gene whose mRNA is fully spliced and transported independently of Rev. Second, the drugs reduced the accumulation of unspliced and spliced RNA from pNL4-3-LucE- to approximately equal extents, and the decrease in RNA accumulation occurred in both nuclear and cytoplasmic compartments (FIG. 4). Third, the drugs inhibited RNA expression from the rev-minus molecular clone pMRev(−) (FIG. 4), and from several deletion constructs that lack rev (FIG. 5).

It is therefore concluded that Rev is not involved in the effects of CPX and DEF reported here. Similarly, CPX and DEF inhibited the expression of p24, which is translated from incompletely spliced gag mRNA, to about the same extent as FF, generated from spliced mRNA (FIG. 3D).

Role of eIF5A

eIF5A-1 depletion by siRNA reduced HIV-driven gene expression in a manner that was not additive with the action of CPX and DEF. DOHH knockdown by siRNA did not significantly impair HIV gene expression in 293T cells, but expression from the viral promoter was reduced by ˜50% in HeLa cells. Knockdown of DHS or of eIF5A-1 in HeLa cells elicited similar effects. Although other drug actions (including the inhibition of other hydroxylases) are not excluded, these findings strengthen the view that the sensitivity of HIV-1 to CPX and DEF results at least in part from their action on eIF5A maturation.

Antiviral Activity of Ciclopirox and Deferiprone

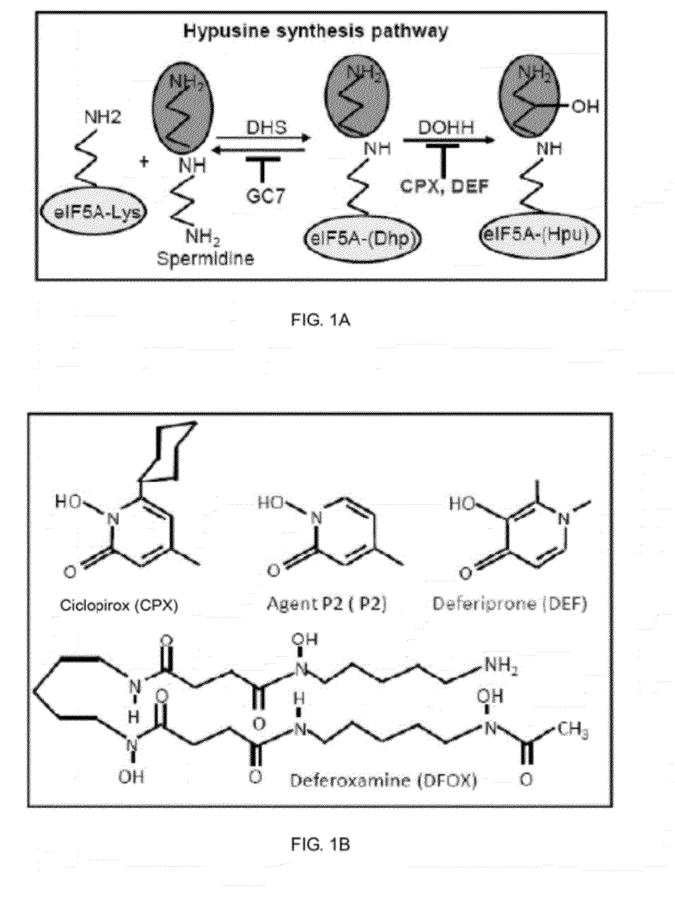

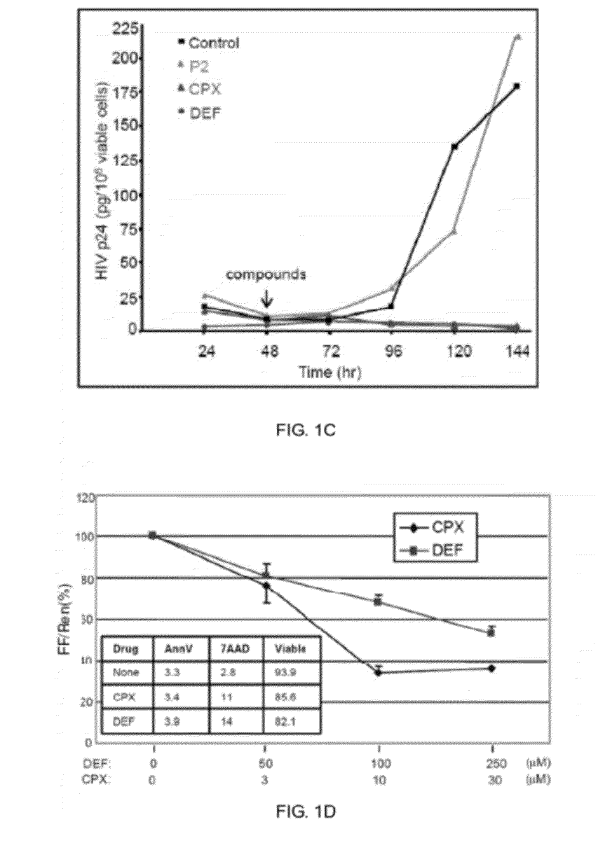

To examine the effect of CPX and DEF on HIV-1 propagation, uninfected PBMCs from healthy donors were co-cultured with HIV-infected PBMCs and virus production was monitored by the p24 capture assay. In untreated cultures, p24 was first detected at 96 hr and increased up to 144 hr (FIG. 1C; Control). Addition of CPX and DEF at 48 hr, to 30 μM and 250 μM respectively, reduced p24 to baseline levels. The profound inhibition is due, at least in part, to activation of apoptosis at later stages of infection. These concentrations are within the clinically relevant range and sufficient to block DOHH activity and eIF5A modification (see below). Agent P2, a chelation homolog of CPX (FIG. 1B), did not impede p24 production (FIG. 1C). These findings indicated that the inhibition of HIV replication by CPX and DEF could be due to inhibition of DOHH and eIF5A maturation.

293T cells were selected as a model system to explore the relationship between the drugs, eIF5A and HIV gene expression. These cells efficiently transcribe HIV-1 genes from molecular clones as well as subviral constructs, allowing for early detection of changes in HIV gene expression. To establish the system, assays were carried out to examine the effect of CPX and DEF on the expression of firefly luciferase (FF) from the HIV-1 molecular clone, pNL4-3-LucE-, engineered to carry the FF gene in place of the viral nef gene. The molecular clone was transfected into 293T cells together with the pCMV-Ren vector that expresses Renilla luciferase (Ren) from the cytomegalovirus (CMV) immediate early promoter as a control for transfection efficiency and non-specific effects of the compounds. Dual luciferase assays were conducted at 12 hr post-transfection and results are expressed as relative luciferase activity (FF:Ren). As shown in FIG. 1D, the drugs repressed expression from the HIV-1 molecular clone in a dose dependent fashion. Long-term drug exposure leads to pleiotropic effects including apoptosis, but marginal 293T cell death was observed within 24 hr using these concentrations of CPX and DEF (FIG. 1D, inset). Assays therefore were carried out to characterize the action of CPX and DEF on eIF5A and HIV gene expression in 293T cells during the first 12 to 24 hr of drug treatment.

Drug Effects on eIF5A and DOHH

To examine the effect of the drugs on the synthesis of modified eIF5A, 293T cells transfected with a FLAG-tagged eIF5A expression vector were simultaneously treated with CPX or DEF. FLAG-eIF5A was monitored using NIH-353 and anti-FLAG antibodies (FIG. 2A,B). The NIH-353 antibody reacts preferentially with post-translationally modified eIF5A. CPX reduced the appearance of mature eIF5A over the 3-30 μM concentration range, while DEF was effective at 200-400 μM. The drugs did not alter the expression of actin. Comparable results have been obtained in other cell types by spermidine labeling of eIF5A. In addition to the CPX homolog Agent P2, deferoxamine (DFOX; Desferal™) was used as a control compound. DFOX, a metal-binding hydroxamate like CPX and Agent P2 (FIG. 1B), is a globally used medicinal iron chelator that does not inhibit HIV-1 infection. In contrast to CPX and DEF, P2 and DFOX had little or no effect on the appearance of mature FLAGeIF5A (FIG. 2A,B), indicating that the ability to chelate iron is insufficient to inhibit DOHH and the maturation of eIF5A. None of these compounds reduced the overall expression of the FLAG-eIF5A protein detectably (FIG. 2C), ruling out general inhibitory effects on gene expression. Based on these results, 30 μM CPX and 250 μM DEF were used for subsequent experiments.

eIF5A forms tight complexes with its modifying enzymes. Unmodified eIF5A (lysine-50) immunoprecipitates with DHS and deoxyhypusyl-eIF5A interacts with DOHH in vitro. It was discovered that the deoxyhypusyl-eIF5A:DOHH complex formed in vivo can be detected by immunoprecipitation from cell extracts. Taking advantage of this finding, the effects of the drugs on the enzyme-substrate interaction were tested. FLAG-eIF5A was expressed in 293T cells and complexes that immunoprecipitated with anti-FLAG antibody were immunoblotted and probed with antibodies against DOHH. Endogenous DOHH co-immunoprecipitated with FLAG-eIF5A, and this association was largely prevented by treatment with CPX or DEF (FIG. 2D, top panel). Consistent with their inability to inhibit eIF5A maturation, neither P2 or DFOX prevented the formation of the eIF5A:DOHH complex. As a further control, the DHS inhibitor GC7 was included in this assay. No DOHH was associated with FLAG-eIF5A in the presence of GC7 because it prevents the synthesis of deoxyhypusyl-eIF5A. As expected, none of the compounds affected the immunoprecipitation of FLAG-eIF5A (FIG. 2D, middle panel) or the expression of endogenous eIF5A (FIG. 2D, bottom panel). Reciprocally, the interaction between endogenous eIF5A and tagged DOHH was inhibited by CPX and DEF (FIG. 2E, right). Similarly, the interaction of endogenous eIF5A with tagged DHS was inhibited by GC7 (FIG. 2E, left) but resistant to CPX and DEF. It is concluded that CPX and DEF, but not P2 or DFOX, target DOHH and inhibit its interaction with its substrate, deoxyhypusylelF5A.

Inhibition of Gene Expression from HIV-1 Molecular Clones

To explore the mechanism whereby CPX and DEF inhibit HIV gene expression, the specificity of their effect on the expression from the pNL4-3-LucE- molecular clone was examined. Exposure to CPX and DEF repressed expression from the HIV-1 molecular clone by ˜50%, as shown above (FIG. 1D), whereas P2 and DFOX were ineffective (FIG. 3A). The drugs had no effect on CMV-driven Renilla luciferase expression. Similar results were obtained in transfected Jurkat T cells (FIG. 3B). RNase protection assays (RPA) showed that the inhibition of luciferase activity by DEF (FIG. 3C) or CPX was reflected in decreased accumulation of FF mRNA, while no change was observed in the accumulation of Ren mRNA from the CMV promoter. Thus, the drugs specifically inhibited luciferase expression from the HIV-1 molecular clone at the RNA level.

Both CPX and DEF also inhibited HIV p24 expression from the molecular clone by ˜60% whereas DFOX had no effect (FIG. 3D). Next, the effects of CPX and DEF on viral mRNA expression were examined. The sensitivity of FF expression from pNL4-3-LucE- to these drugs suggested that the inhibition of RNA accumulation is independent of Rev since the FF sequences are substituted into the nef gene which gives rise to spliced mRNA. To determine whether the action of CPX and DEF is exerted at the level of the accumulation, splicing or nucleo-cytoplasmic distribution of HIV RNA, pNL4-3-LucE- was transfected into 293T cells and monitored spliced and unspliced HIV RNA after drug treatment. RNase protection assays were carried out using a probe complementary to the 5′ region of all HIV-1 transcripts (Zheng et al. Nat Cell Biol 2003, 5:611-618). The probe spans the major splice donor site so two sizes of protected fragments are generated: unspliced RNA protects an RNA fragment 50 nucleotides (nt) longer than that from spliced RNAs (FIG. 4A). CPX and DEF, but not P2, reduced the level of both spliced and unspliced RNAs by ˜50% (FIG. 4B). A similar reduction was observed in both the cytoplasmic and nuclear fractions. In contrast, the production of Renilla luciferase RNA driven by the CMV promoter was unchanged in the nucleus and cytoplasm after drug treatment (FIG. 4B). Thus, the drugs cause an overall inhibition in HIV RNA expression as early as 12 hr after drug addition.

These experiments did not disclose a significant effect on the splicing or export of viral RNA as a result of treatment with CPX or DEF. Because previous reports indicated that modified eIF5A is involved in the Rev-dependent export of unspliced and underspliced HIV-1 RNAs, assays were carried out to examine whether the drugs affect the splicing or export of viral RNAs mediated by Rev. The rev-defective molecular clone pMRev(−) contains the entire HIV-1 genome but Rev expression is prevented by substitutions in its initiation codon. To compare the inhibitory effect of CPX and DEF in the presence and absence of Rev, cells were transfected with pMRev(−), either with or without a Rev expression vector, and RNA was analyzed by RPA as above. As expected, in the absence of Rev there was very little unspliced RNA in the cytoplasm although substantial levels were present in the nucleus, and Rev expression increased the level of unspliced RNA in the cytoplasm (FIG. 4C). Treatment with CPX or DEF reduced the levels of both spliced and unspliced RNAs in the nucleus and cytoplasm by 2-3 fold irrespective of the presence or absence of Rev (FIG. 4C). Similar data were obtained in COS7 cells. These results indicate that the drugs inhibited HIV-1 RNA accumulation by a mechanism independent of Rev-mediated viral RNA splicing and export, consistent with the inhibition of FF expression from pNL4-3-LucE-(FIG. 3).

### Genetic Requirements for Drug Sensitivity

The data obtained with pMRev(−) excluded involvement in the drug responses of the env mutation, nef deletion and FF gene insertion in pNL4-3lucE-, as well as the rev gene. To search for viral elements that confer sensitivity to CPX and DEF in these short-term experiments, a series of truncations of the HIV-1 genome were generated. Unique restriction sites were exploited to delete major open reading frames from pNL4-3-lucE- (FIG. 5A). Compared to the parental clone (construct II), construct III has a deletion of nt 1506-5784 affecting gag, pol and vif, while construct IV lacks nt 5784-8476 eliminating the expression of vpr, vpu, tat, rev and env. These two deletions encompass nearly all of the viral coding sequences. Nevertheless, FF expression from these constructs was inhibited _{—}50% by CPX and DEF within 12 hr (FIG. 5B). (Note that Tat-deficient constructs were complemented by cotransfection of a Tat expression vector in these assays.) Subsequently, construct V was produced by deleting all the open reading frames except for luciferase from the nef coding region. Drug inhibition of this construct, which retains only ˜1,967 nt of viral sequence, was also _{—}50% (FIG. 5).

All of these constructs have two intact LTRs, derived from the 5′ and 3′ ends of the molecular clone. When the 3′-LTR of construct V, which contains the HIV-1 poly(A) signal, was replaced by a poly(A) signal from SV40 in construct VI, expression was still inhibited ˜50% by CPX and DEF (FIG. 5) indicating that the 3′-LTR is not the determining feature. Construct VI contains 321 nt of env as well as the nef ATG, but these sequences can also be excluded as demonstrated by construct VII (pLTR-FF) in which the 5′ LTR is the only segment derived from HIV (FIG. 5). By contrast, expression from pCMV-FF (construct I) was unaffected by CPX and DEF (FIG. 5), consistent with the above findings with pCMV-Ren (FIGS. 3 and 4). Thus, the inhibition of gene expression by both drugs is specific for the HIV 5′-LTR.

CPX and DEF Inhibit Transcription Initiation at the HIV-1 Promoter

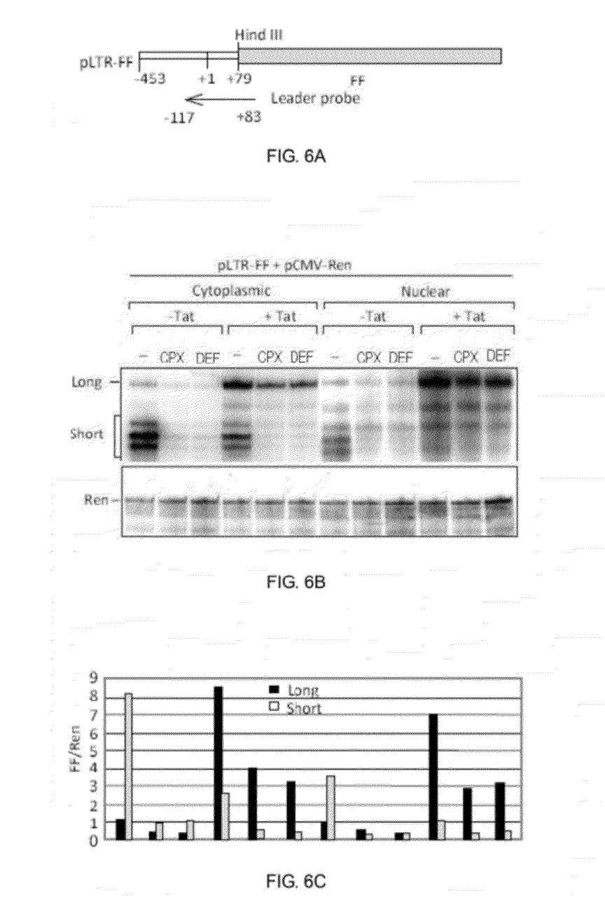

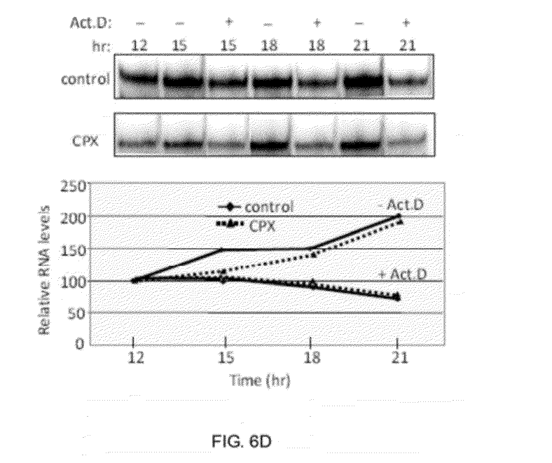

Results of the deletion analysis implied that sensitivity to the drugs is conferred by the promoter or another feature in the HIV-1 LTR. A conspicuous feature of HIV transcription is its dependence on the viral Tat protein and the cellular complex P-TEFb (positive transcription elongation factor b) that cooperate to ensure processive transcription and the formation of long viral transcripts. To determine whether the drugs inhibit at the elongation step, assays were carried out to examine their effect on HIV-1 transcripts generated in COST cells co-transfected with pLTR-FF and pCMV-Ren in the presence or absence of a Tat expression vector. Nuclear and cytoplasmic RNA was analyzed in RNase protection assays using a probe complementary to the promoter-proximal region of HIV transcripts (FIG. 6A). Short fragments corresponding to RNA of ˜55-59 nt predominated in the absence of Tat, whereas longer fragments of ˜83 nt accumulated in its presence (FIG. 6B). Similar observations were made in the cytoplasm and nucleus. Treatment with CPX and DEF diminished both signals by 50-80% irrespective of the presence or absence of Tat (FIG. 6B,C). These results argue against a specific effect at the level of HIV transcription elongation.

To examine the possibility that the drugs decrease the stability of RNA transcribed from the HIV promoter, cells transfected with pLTR-FF were incubated in the presence or absence of CPX. Actinomycin D was added to some cultures 12 hr later to block further transcription, and FF RNA was monitored by RPA at intervals thereafter (FIG. 6D, top panel). FF RNA levels were quantified and normalized to the levels at 12 hr (FIG. 6D, bottom panel). FF RNA continued to accumulate in the absence of actinomycin D but declined in its presence. The rate of RNA decay was not affected by the presence of CPX (FIG. 6D). Similar results were obtained with DEF (data not shown). It is therefore concluded that the drugs inhibit HIV-1 transcription initiation.

Inhibition of eIF5A Production Reduces HIV Gene Expression

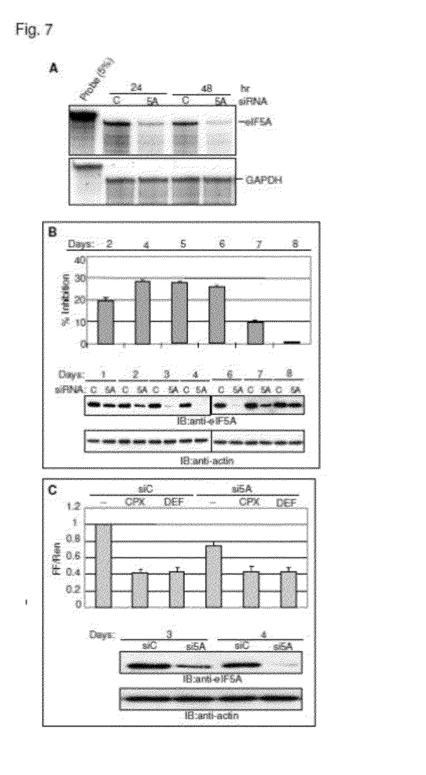

The findings described to this point establish a correlation between inhibition of eIF5A modification and inhibition of HIV-1 gene expression. To examine the effect of eIF5A hydroxylation directly, assays were carried out to deplete DOHH by RNA interference. No significant effect on eIF5A modification or HIV gene expression was detected, however, probably because the level of DOHH was not reduced below 60% (data not shown). Attention was therefore turned to siRNA directed against eIF5A-1 itself. Compared to non-targeted control siRNA, eIF5A-1 siRNA reduced the level of its cognate RNA by ˜80% at 24 hr (FIG. 7A). The eIF5A protein level declined more gradually, consistent with its long half-life, to a minimum of ˜30% of control levels at 96 hr post-siRNA transfection (FIG. 7B). GAPDH mRNA and actin protein levels were unchanged, indicating that eIF5A siRNA does not exert a broad deleterious effect in these cells (FIG. 7A, B).

eIF5A knockdown reduced gene expression from the HIV-1 molecular clone by ˜30% between 4 and 6 days post-transfection (FIG. 7B, top panel). Although the magnitude of this effect was relatively modest, presumably because of incomplete depletion of eIF5A, two observations attest to its importance. First, the inhibition of HIV-driven gene expression correlated with eIF5A knockdown and recovery (FIG. 7B, lower panel) indicating that targeted reduction of eIF5A expression correlates with inhibition of HIV-driven gene expression. Second, the effects of the drugs and siRNA were not additive. When cells transfected with siRNA for 3 or 4 days were exposed to the drugs for the last 12 hr of this period, eIF5A knockdown did not elicit a further inhibition of HIV-1 gene expression (FIG. 7C). These observations are consistent with the drugs functioning in the hypusine pathway to inhibit HIV-1 RNA accumulation.

Ciclopirox and deferiprone, two clinically used drugs, block HIV-1 infection. In model systems, the drugs inhibit the enzyme DOHH required for maturation of eIF5A and repress expression from the HIV-1 promoter at the level of transcription initiation.

### Example 2

### Drug-Induced Reactivation of Apoptosis Abrogates HIV-1 Infection

CPX and DEF Trigger Apoptosis in H9 Cells Via the Intrinsic Pathway

A common mode of action of CPX and DEF was investigated via the induction of apoptosis in HIV-infected cells.

First, whether both drugs elicit apoptosis in H9-HIV cells was determined. The cells were exposed to CPX or DEF, and annexin-V binding, cell diameter and cell survival were assayed in a time-dependent manner. By 24 hr, drug treatment elicited a ˜5-fold increase in the percentage of the 7-AAD-negative cell population capable of binding annexin-V to exposed membrane phospholipid phosphatidylserine, with little further increase at 48 hr (FIG. 8A). Both drugs elicited a decrease in mean cell diameter of ˜15% within 24 hr and ˜30% within 48 hr, indicating volume constriction characteristic of apoptosis but not necrosis (FIG. 8B). Concomitantly, cell survival decreased ˜2 fold at 24 hr and ˜5 fold at 48 hr (FIG. 8C). CPX and DEF exerted similar effects with similar kinetics on these apoptotic indicators, indicating that the drugs both trigger apoptosis in this T lymphocytic cell line chronically infected with HIV-1.

Collapse of the mitochondrial membrane potential, Δψ, is an early event in apoptotic death triggered via the intrinsic pathway, leading to proteolytic activation of initiator and effector caspases including caspase-3. One consequence of caspase-3 activation is the cleavage of poly (ADP-ribose) polymerase (PARP), resulting in the accumulation of an 89-kDa PARP fragment indicative of nuclear proteolysis. Therefore, the Δψ and PARP status of H9 and H9-HIV cells were monitored. Flow cytometric analysis showed that both the collapse of Δψ and the cleavage of PARP were attenuated in HIV-H9 cells relative to H9 cells (FIG. 8D). Specifically, Δψ collapse was about half as frequent in HIV-H9 cells as in uninfected H9 cells, and approximately one-third as many cells were positive for PARP cleavage in HIV-H9 cell cultures as in uninfected H9 cultures. These data indicate that apoptosis in H9 cells is triggered via the intrinsic pathway and is attenuated by HIV-1 infection.

### Drug-Mediated Reversal of Resistance to Apoptosis in HIV-Infected Cells

Next the effect of the drugs on apoptosis in H9 and H9-HIV cells was examined. Both CPX and DEF increased the collapse of Δψ in a manner that was dose-dependent and accentuated by viral infection (FIGS. 8E and F). At the standard drug concentrations used in this study (30 μM CPX; 200 μM DEF), infected cells displayed significantly increased collapse of Δψ compared to uninfected cells. Furthermore, the H9-HIV cultures exhibited enhanced Δψ collapse even at low drug concentrations (5 and 15 μM CPX; 50 and 100 μM DEF), whereas higher concentrations were required in uninfected H9 cells (30 μM CPX; 200 μM DEF). Thus, exposure to CPX or DEF counteracted the HIV-mediated reduction of Δψ collapse and rendered infected cells more susceptible than uninfected cells to an early step in drug-induced apoptosis.

To determine whether the differential effects of the drugs extend into late apoptosis, PARP fragmentation in H9 and H9-HIV cells was measured. CPX caused an ˜8-fold increase in H9-HIV cells positive for 89-kDa PARP, compared to a ˜2 fold increase in H9 cells (FIG. 9A). By 24 hr, twice as many cells stained positive for 89-kDa PARP in the infected cultures as in uninfected cultures (27% compared to 14%; FIG. 9B). Furthermore, the fluorescence intensity was approximately one order of magnitude higher in the presence of HIV-1 (FIG. 9B). These differences persisted after 48 hr of CPX treatment (FIG. 9C), and DEF gave similar but less pronounced effects. It is concluded that the retroviral suppression of initiation and execution of apoptosis is reversed by the drugs and transformed into enhanced susceptibility of HIV-infected cells to apoptosis.

### Cellular and Viral Protein Levels During CPX-Induced Apoptosis

The enhancement of Δψ collapse indicated that the drugs might repress antiapoptotic proteins that stabilize Δψ, in particular Bcl-2. CD4+ T cells isolated from infected individuals have increased Bcl-2 levels compared to uninfected lymphocytes and some reports implicate HIV-1 Tat in preventing apoptosis in persistently infected cells by inducing the transcription of Bcl-2. Since CPX blocks HIV-1 gene expression, Bcl-2 expression in H9 and H9-HIV cells was determined. After treatment with CPX for 24 hr, ˜35% of cells stained positive for Bcl-2, irrespective of infection (FIG. 10A). Although uninfected cells displayed a modest dose-dependent decrease in Bcl-2 content, contrasting with a slight increase in infected cells, neither of these measures achieved statistical significance (FIG. 10B). DEF gave similar results (data not shown). These data do not support a role for Bcl-2 suppression in the drug-induced enhancement of apoptosis in H9-HIV cells, in accord with conclusions drawn from a study of other agents that cause apoptosis in HIV-infected cells. Retroviral proteins can also control apoptosis. Therefore, assays were carried out to measure the response of individual retroviral proteins in H9-HIV (FIG. 10C) to the shut-down of the HIV-1 promoter by CPX. CPX reduced intracellular p24 by 30% within 24 hr, and by 70% within 48 hr, consistent with results in 293T cells. Intracellular Tat was similarly reduced. Rev was only marginally affected, however. This differential susceptibility mirrors their dissimilar intracellular stabilities: the half-life of p24 and Tat is ˜3 hr while that of Rev is at least 16 hr. Paradoxically, the levels of Vpr increased by ˜15% within 24 hr and ˜70% within 48 hr, suggesting a degree of autonomy from transcription-dependent synthesis consistent with its previous reports. DEF elicited a similar response, sparing Rev, decreasing p24 and Tat, and increasing Vpr. Vpr induces apoptosis via caspase-3 activation. In H9-HIV cultures, the increase in Vpr paralleled that of active caspase-3 (FIG. 10C). Vpr-driven cell death is characterized by many of the parameters recorded above—increased annexin binding, changes in cell size and volume, Δψ collapse, and PARP cleavage—and DNA fragmentation discussed below. These observations indicated a functional involvement of Vpr, and possibly Tat, in CPX-induced apoptotic death of HIV-1 infected cells.

### Structure-Activity Relations

Both CPX and DEF are hydroxypyridinones with vicinally positioned oxygen atoms that mediate bidentate interaction with metal ions, in particular Fe³⁺ (FIG. 11A). To evaluate the effect of drugs on the intracellular iron pool and on HIV-1 gene expression, transient expression assays in human 293T cells were exploited. Bioavailable intracellular iron was measured using a construct in which an iron-responsive element (IRE) controls luciferase expression, and HIV-specific transcription was assayed in parallel using an HIV-1 molecular clone that expresses luciferase reporter under the direction of the retroviral promoter. CPX and DEF were compared with the medicinal chelator deferoxamine (DFOX) and with the CPX fragment Agent P2, which consists of the metal-binding 1,2-HOPO domain of CPX but lacks the cyclohexyl moiety (FIG. 11A).

CPX and DEF reduced intracellular iron by 80% and ˜60%, respectively (FIG. 11B, hatched bars), and decreased HIV expression by 40-50% as expected (FIG. 11B, filled bars). DFOX, tested at the peak concentration achievable in patients (15 μM), also reduced intracellular iron by 80% (FIG. 11B). DFOX had no effect on HIV-driven gene expression, however, indicating that depletion of bioavailable intracellular iron does not translate into antiretroviral activity. This conclusion is consistent with the failure of DFOX to suppress HIV-1 gene expression and replication in culture and to prevent disease progression and death in HIV-1 infected patients. DFOX is an effective inhibitor of iron-containing protein hydroxylases only at supraclinical concentrations. As predicted from its structure, Agent P2 displays a CPX-like ability to form bidentate chelates with Fe³⁺ and tris(N-hydroxypyridinone ligand) complexes, but it had no effect on either IRE-dependent or HIV-encoded luciferase expression (FIG. 11B). Furthermore, Agent P2 failed to inhibit eIF5A hydroxylation in vivo or in vitro. These data suggest that Agent P2 lacks lipophilicity required for cell entry and DOHH inhibition, and indicate that the cyclohexyl moiety of CPX is required for antiretroviral activity.

The relationship between iron chelation and apoptosis was assessed using the TUNEL assay to measure DNA fragmentation. DEF induces this late apoptotic event in HIV-expressing ACH-2 cells. Similarly, CPX triggered DNA fragmentation in H9-HIV cells in a dose-dependent manner with a steep, almost 10-fold increase in TUNEL-positive cells between 10 and 20 μM CPX that leveled off at 40 μM (FIG. 11C). In contrast, DFOX did not enhance DNA fragmentation even at 20 μM, the maximum achievable in human plasma (FIG. 11C). It was noted that the dose-dependencies for TUNEL reactivity and inhibition of eIF5A hydroxylation were indistinguishable: Apoptotic DNA fragmentation correlated positively with accumulation of non-hydroxylated eIF5A (r=+0.995; P>0.01) and negatively with depletion of hydroxylated eIF5A (r=−0.996; P>0.01) (comp. FIGS. 4C and 4D). Accumulation of non-hydroxylated eIF5A, i.e. the lysyl- or deoxyhypusyl-intermediate, is apoptogenic in several cell lines.

Evidently the antiretroviral activities of CPX and DEF in HIV-infected cells require more than global iron chelation. Specifically, apoptosis and the inhibition of retroviral gene expression by these drugs is not mediated simply via the depletion of extracellular or intracellular ferric ions, or of other metal ions chelated by the drugs.

Inhibition of Acute Viral Infection and Activation of Apoptosis in Infected PBMC Cultures

To examine the antiretroviral action of CPX in a clinic-derived model, PBMCs infected with patient isolates of HIV-1 were used. Naïve freshly isolated PBMCs from a single donor were infected by co-cultivation with HIV-infected, HLA-nonidentical PBMCs at an initial uninfected/infected cell ratio of 10:1. CPX was added and cells were maintained and monitored for six days. Half of each culture supernatant was replenished daily with CPX-containing or CPX-free medium as appropriate. In control cultures, robust infection invariably occurred during this period, achieving p24 and viral RNA copy levels and a p24/viral copy ratio similar to those reported in patients, although the kinetics of infection varied depending on the particular PBMC donor/HIV isolate combination.

For the donor/isolate combination shown in FIG. 12, p24 formation rapidly increased 96 hr after inoculation with HIV-infected PBMCs. Addition of CPX at 48 hr completely inhibited p24 production and doubling the CPX concentration to 60 μM did not further increase this suppressive effect compared to controls without drugs (FIG. 12A). Correspondingly, CPX inhibited the production of HIV-1 RNA (FIG. 12B), indicating that the drug completely suppressed the establishment of productive Infection. Agent P2, the chelation homolog of CPX, did not curtail the accumulation of p24 or HIV-1 RNA (FIGS. 12A and B), consistent with its lack of effect on HIV-1 gene expression in H9-HIV cells (FIG. 11B). The delay-insensitivity of the antiretroviral activity of CPX in PBMCs argues against interference with early events such as retroviral binding to cell receptors or fusion with the target cell membrane. Agents that block these events fail to suppress acute HIV-1 infection if delayed by as little as 2 hr post-inoculation.

The induction of apoptosis was analyzed with donor/isolate combinations that displayed rapid emergence of productive infection. In such cultures, the protein and nucleic acid indices of active retroviral infection rose within 72 hr after inoculation with infected PBMCs (FIGS. 13A and B). The increases in retroviral p24 and RNA occurred in a synchronous manner (r=+0.92, P<0.01). CPX, whether added at the time of inoculation (data not shown) or 12 hr later (FIGS. 13 A and B), inhibited HIV-1 replication. HIV-1 p24 remained undetectable throughout six days of incubation (FIG. 13A), and extracellular HIV-1 RNA did not increase at any time over the initial infectious dose, whereas it increased >100 fold in HIV-1 exposed untreated cultures (FIG. 13B). Using the TUNEL assay for DNA fragmentation, <10% of cells in infected or uninfected untreated cultures were apoptotic 144 hr after inoculation (FIG. 13C). This population increased to 24.1% (±3.2%) of cells in CPX-treated uninfected cultures. By contrast, it rose dramatically to 71.8% (±8.8%) of CPX-treated cells exposed to HIV-1 (P=0.009) (FIG. 13C). Similar to H9 cells (FIG. 9A), CPX roughly doubled the proportion of TUNEL-positive PBMCs in uninfected cultures but caused a ˜10-fold increase in HIV-infected cultures. Apoptosis emerged only after 120 hr in infected cultures (termed Phase II, FIG. 13) while productive PBMC infection in untreated cultures reached a maximum earlier, within 72 hr (Phase I). Thus, untreated infected PBMCs displayed marked HIV-1 expression in Phase I and minimal apoptosis in Phase II, whereas HIV-1 exposed CPX-treated PBMCs showed no HIV-1 expression in Phase I but extensive apoptosis in Phase II. These observations are consistent with a reduction in the apoptotic threshold of infected cells if the expression of critical HIV-1 genes is blocked.

To further assess the cellular effects of CPX, the secretion of interferon (IFN)-γ and interleukin (IL)-10 was measured. These cytokines are pivotal regulators of the immune response and both are responsive to HIV-1 infection. IFN-γ, a Th1 cytokine that activates antiviral defenses, is elevated early in infection and decreases with disease progression. IL-10, a Th2 cytokine that limits immune system activity, increases markedly as CD4 counts fall. The secretion of these cytokines was measured in infected, CPX-treated or untreated PBMC cultures before and during the non-apoptotic Phase I. In HIV-exposed untreated cultures, the levels of IFN-γ and IL-10 increased after 72 hr by ˜14- and ˜4-fold, respectively (FIGS. 13D and E), paralleling the rise of viral parameters (FIGS. 13A and B). CPX treatment abolished the HIV-induced cytokine boost, irrespective of whether the drug was added at the time of inoculation (FIGS. 13D and E) or 12 hr later (data not shown). The absent response of both cytokine biomarkers to the presence of HIV-1 in drug-treated cultures concurred with the inhibition of virological indices (FIGS. 12A and B). It is concluded that CPX blocks the acute infection of freshly isolated PBMCs by patient isolates of HIV-1 to such an extent that the innate cytokine response is squelched.

### Termination of Infection in Drug-Treated PBMCs

Next, assays were carried out to examine whether CPX can control established HIV-1 infection in primary cells. Long-term PBMC cultures were employed as a model for on-going, self-sustaining HIV-1 production. As before, infection was initiated by exposure to patient isolate-infected cells. To emulate the bulk flow of susceptible cells from a generative into an infective compartment that occurs in vivo, a replenishment protocol was followed. Freshly isolated uninfected primary cells were infused into the infected cultures at regular intervals during multi-month monitoring of viral parameters. HIV-1 RNA reached the range of 10⁶ copies/ml within a week of patient isolate inoculation, and this robust infection was sustained for >3 months (FIG. 14; open squares).

The introduction of CPX on day 7, adjusted daily to maintain a constant level of 30 μM, reduced the levels of p24 and viral RNA to the limit of detectability (FIG. 14; closed circles and triangles, respectively). The inhibition of p24 occurred rapidly while the decline in HIV-1 RNA levels, though eventually spanning four orders of magnitude, was delayed. This finding was attributed to the broad dynamic range of the PCR-based RNA assay, compared to the relatively narrow range of the ELISA-based p24 assay, and possibly to the packaging of RNA into apoptotic bodies that protect against degradation by RNase. Mathematical modeling indicated that the viral RNA level decreases more slowly than the rate calculated for depletion by medium replenishment, arguing against a protocol-related artifactual decline and for the presence of a dwindling pool of HIV-1 RNA in the medium. The inability to detect either viral protein (after day 21) or RNA (after day 38) indicated that CPX-driven apoptotic depletion of infected cells might have driven the virus to the point of eradication.

To test this interpretation and to determine whether infected cells generating infectious virus persisted, the possibility of viral resurgence was examined. Cultures were maintained for an extended off-drug inspection period and monitored for the re-emergence of HIV-1 RNA (Phase III). Strikingly, HIV-1 infection did not rebound after discontinuation of either drug (asterisk in FIG. 14) during an off-drug inspection period extending to 12 weeks. Similar results were obtained in repeated experiments with shorter off-drug inspection periods (data not shown) and with DEF (Saxena et al., in preparation). Thus, drug treatment for ˜1 month repressed viral replication and the virus did not rebound over the subsequent ˜3 months in the absence of drug. It is concluded that the robust and self-perpetuating HIV-1 infection in these mixed lymphocyte cultures behaved as if biologically silenced by the drugs.

### Lack of Apoptosis Induction in Vaginal Mucosa and Human Epithelial Cells

In the experiments reported here, CPX and DEF enhanced apoptotic indices in HIV-infected cells, and also in uninfected cells albeit to a significantly smaller degree (FIGS. 8E and F; FIG. 13C). DEF is a systemically active drug, and CPX preparations are in direct, prolonged contact with human skin or epithelia. These drugs might be employed to block infection via the genital mucosa, the main route of HIV-1 transmission. Their effects were therefore studied in two assay systems established as predictive for toxicity to human genital mucosa. The gynecological preparation of CPX (1% Batrafen Vaginalcrème™ [Sanofi-Aventis]), containing 28.8 mM CPX, was tested in a mouse model for epithelial barrier integrity. Since no topical DEF preparation was available, freshly dissolved DEF was tested in a human mucosal cell culture model.

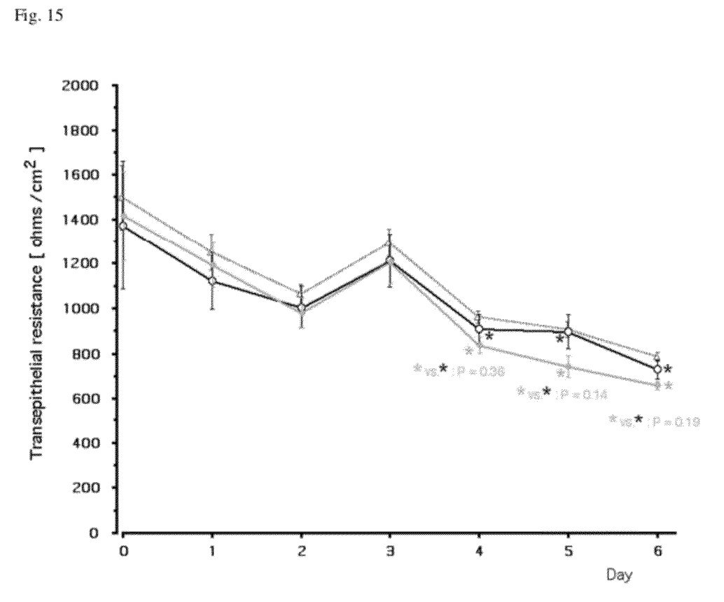

DEF was analyzed for its effect on the trans-epithelial resistance (TER) displayed by confluent human ECC-1 cells linked by tight junctions. After the cells achieved maximal epithelium-like barrier function, DEF was added and TER measurements were recorded over the following six days. The medicinal chelator DFOX (20 μM) was tested as a control. Exposure to DEF did not reduce TER beyond the spontaneous decay observed in drug-free controls, nor did exposure to DFOX (FIG. 15). Efficient chelation of intra- and extracellular iron by DEF or DFOX (FIG. 11B) therefore does not damage ECC-1 cells. In particular, DEF does not degrade the physicochemical barrier formed by a single layer of polarized endometrial cells at the concentration that inhibits HIV-1 replication.

CPX was evaluated both functionally and histologically for effects on murine vaginal mucosa. Intravaginal application of Batrafen Vaginalcrème™ for four consecutive days did not increase susceptibility to vaginal infection by low- or high-dose challenges with herpes simplex virus type 2 (HSV-2). In the high-dose HSV-2 group, 8 of 10 animals became infected, whether CPX-treated or not; in the low-dose HSV-2 group, one of the untreated and none of the CPX-treated animals became infected. This lack of a gross effect on the protective function of the cervicovaginal mucosa was corroborated by histological examination. CPX exposure did not disturb the medroxyprogesterone-induced surface-lining layer of living mucinous cells in these mice, nor did the drug disrupt the underlying layers of squamous epithelial cells (FIGS. 16A and B). Notably, CPX did not trigger apoptosis in the epithelial or subepithelial compartments, as assessed by immunohistochemical detection of active caspase-3. This protease locates to the nucleus after induction of apoptosis. Reactivity to anti-active caspase-3 occurred in a typical punctate pattern, highlighting the nuclei of cells undergoing apoptosis in tissue, as shown for human neonatal thymus (FIG. 16C) and mouse ovarian follicles (FIG. 16D). The nuclei of mucinous and squamous epithelial cells in CPX-exposed vaginal mucosa did not react with anti-active caspase-3, and their faint cytoplasmic hue did not differ from untreated mucosa (FIGS. 16A and B).

Neither CPX nor DEF gave evidence of apoptotic or other toxic effects on uninfected epithelia when applied at or above the concentrations that ablate HIV-1 infected cells (FIGS. 8, 9, 11 and 13) and suppress de novo infection of PBMCs by viral isolates (FIGS. 12-14).

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated herein in their entireties.

What is claimed is:

1. A method of identifying a compound for treating an infection with a virus, the method comprising:
mixing a test compound with a first plurality of cells in a medium for a first period of time, the cells being infected with the virus;
culturing the cells for a second period of time; and
determining the activity level of the promoter of the virus in the cells;
wherein the activity level in the presence of the test compound, if lower than that in the absence of the test compound, indicates that the test compound is a candidate for treating the infection with the virus.

2. The method of claim 1, wherein the culturing step comprises (i) removing the test compound from the medium and (ii) maintaining the cells for the second period of time.

3. The method of claim 1, wherein the determining step is conducted by determining the transcription initiation level.

4. The method of claim 1, wherein the method further comprises evaluating the apoptosis level of the cells and wherein the level of apoptosis in the presence of the test compound, if higher than that in the absence of the test compound, indicates that the test compound is a candidate for treating the infection with the virus.

5. The method of claim 1, wherein the virus is an HIV-1 virus.

6. The method of claim 1, wherein the first plurality of cells are PBMCs.

7. The method of claim 6, wherein the method further comprises evaluating the level of IL-10 or IFN-γ in the medium or cells.

8. The method of claim 1, wherein the method further comprises (a) contacting the first plurality of cells or the medium with a second plurality of cells, and (b) determining the activity level of the promoter of the virus in the second plurality cells.

9. The method of claim 1, wherein the first period of time is 2 hours-1 month.

10. The method of claim 1, wherein the second period of time is up to 3 months.

11. A method of reducing or eliminating HIV-1 rebound subsequent to treatment of an HIV-1 infected subject comprising administering an iron-chelating hydroxypyridinone (HOPO) compound to a subject infected with HIV-1 in an amount and for a time effective to reduce or eliminate the level of HIV-1 virions, followed by discontinuing administration of said iron-chelating hydroxypyridinone, whereby the level of HIV-1 virions remains reduced or eliminated for at least 4 weeks after discontinuation of administration.

12. The method of claim 11, wherein the level of HIV-1 virions remains reduced or eliminated for at least 12 weeks after discontinuation of administration.

13. The method of claim 11, where said time effective to reduce or eliminate the level of HIV-1 virions is 4 weeks.

14. The method of claim 11, wherein the method further comprises administering to the subject an apoptosis inducer.

15. The method of claim 11, wherein the iron-chelating hydroxypyridinone is selected from the group consisting 6-cyclohexyl-1-hydroxy-4-methylpyrid-2(1H)-one (ciclopirox) and 3-hydroxy-1,2-dimethylpyridin-4(1H)-one (deferiprone).

16. An immunogenic composition comprising (i) one or more cells that have been infected with HIV-1; (ii) an iron-chelating hydroxypyridinone compound; and (iii) a pharmaceutically acceptable carrier.

17. The immunogenic composition claim 16, wherein the cells are peripheral blood mononuclear cells (PBMCs).

18. The immunogenic composition claim 16, wherein the composition further comprises an adjuvant.

19. A method of eliciting an HIV-1-specific immune response in a subject, comprising administering to a subject in need thereof the immunogenic composition of claim 16.

20. The method of claim 24, wherein the subject has been infected with HIV-1.

21. A method of increasing resistance to HIV-1 infection in a subject, comprising (i) identifying a subject that has been, or is suspected of having been, or is expected to be, exposed to HIV-1, and (ii) administering to the subject an iron-chelating hydroxypyridinone in an amount and for a time effective to maintain or decrease the IL-10 or IFN-γ level in the subject.

22. The method of claim 21, wherein the method further comprises administering to the subject an apoptosis inducer.

23. The method of claim 21, wherein the method further comprises determining the IL-10 or IFN-γ level in the subject after the administering step.
